(19) United States
(12) Patent Application Publication
(10) Pub. No.: US 2015/0272696 A1
(43) Pub. Date: Oct. 1, 2015

(54) SURGICAL SYSTEM

(71) Applicant: INLINE ORTHOPAEDICS PT LTD, West End, Queensland (AU)

(72) Inventors: Don Fry, Portsmouth (AU); David John Cooper, London (GB); David John Wadley, Brisbane (AU); Martin John Ford, Samford (AU)

(21) Appl. No.: 14/437,805

(22) PCT Filed: Sep. 18, 2013
(86) PCT No.: PCT/AU2013/001069

§ 371 Date: Apr. 22, 2015

(30) Foreign Application Priority Data
Oct. 26, 2012 (AU) 2012904715

(51) Int. Cl. A61B 19/00; A61B 17/16; A61B 5/11; A61F 2/46

(52) U.S. Cl. CPC A61B 19/5244; A61F 2/4609; A61B 17/1666; A61B 5/1121; A61F 2/4657; A61B 2019/5229; A61B 2019/5248; A61B 2019/5483

(57) ABSTRACT

The present invention relates to, inter alia, a surgical system for monitoring the orientation of a surgical device relative to a patient's anatomy, the system comprising: a. A patient sensor for sensing the orientation of the patient's anatomy; b. An orientation sensor for sensing the orientation of a surgical device; and c. A monitor for monitoring the orientation of the surgical device relative to the sensed patient's anatomy.

### TECHNICAL FIELD

The present invention generally relates to medical technology, especially to surgery, surgical systems and surgical methods.

### BACKGROUND

It will be clearly understood that, if a prior art publication is referred to herein, this reference does not constitute an admission that the publication forms part of the common general knowledge in the art in Australia or in any other country.

Correct alignment of a prosthetic component is very important in orthopaedic procedures, such as total hip replacement surgery. Optimal alignment may enhance the initial function and long term operability of the prosthetic component. Misalignment of the prosthetic component can result in patient pain and many other potential complications.

Correct alignment of a prosthetic component can be complex, involving multiple steps. Total hip replacement surgery, for example, typically involves: severing the femoral head and dislocating the head of the femur from the acetabulum; reaming the acetabulum to fit an acetabular cup and then inserting the acetabular cup; shaping the femoral canal, and then fitting a prosthetic femoral component into the canal; and fitting the prosthetic femoral component to the acetabular cup. Consequently, to successfully mimic a normal hip joint the acetabular cup and the prosthetic femoral component must be correctly aligned with each other and with the patient's pelvis and femur.

One of the most difficult steps in total hip replacement surgery is correctly aligning the acetabular cup in the acetabulum. For example, in a study spanning 4226 hip replacement surgeries in multiple centres, surgeons were only able to align the acetabular component within an optimal zone in less than 30% of procedures (Langton, D. J. et al. (2011), J Bone Joint Surg[Br] 93-B:164-71). This optimal zone corresponds to a safe zone derived from an earlier work based upon an analysis of serum metal ion results and explants and was defined as 40° to 50° inclination and 10° to 20° anteversion. Consequently, it was concluded that “there is overwhelming evidence to show that surgeons cannot consistently position the acetabular components precisely. Without exception, studies show wide variations in the angles of inclination of the acetabular component and, to an even greater extent, its anteversion.” This is significant as an error in acetabular cup placement of as little as 5° can result in patient complications.

In total hip replacement surgery misalignment of the acetabular cup on the pelvis can result in dislocation of the hip joint, misalignment of the patient's leg, incorrect leg length, decreased joint motion and joint pain. A misaligned acetabular cup on the pelvis may affect the patient's posture, the biomechanics of the lower limbs (for example affecting the range of movement of the hip), and the degree of curvature of the thoracic and cervical spines. The long term effects of a misaligned acetabular cup can include accelerated wear of the components, aseptic loosening of the components and potentially early revision surgery. Furthermore, misalignment may increase the leaching of the prosthesis metal components (for example into the blood stream), which can lead to immune system problems.

It may be possible for surgeons to use various anatomical landmarks on the patient to serve as a guide for correct placement of the acetabular cup during surgery. However, these landmarks generally remain obscured from view during surgery, making it very difficult for surgeons to assess the optimal orientation. It is believed that most surgeons visually estimate the position of the acetabular cup based on its relationship to the trunk of the patient, which is partially obscured with surgical drapes, and decide if it meets the desired orientation before securing it. To our knowledge, specific guidance devices that orient the acetabular cup to the anatomy of the pelvis are not used by most surgeons. Nevertheless, a number of devices and systems have been developed to assist in the optimal placement of a prosthetic component such as an acetabular cup.

For example, International Publication No. WO2010/031111 describes a surgical orientation system for assisting a surgeon to orient a prosthetic component relative to a patient's anatomy. The system operates by attaching an electronic orientation monitor to an implement to which a prosthetic component is attached. A patient is attached to a brace, and the brace defines a reference point relative to the patient's anatomy for calibrating the electronic orientation monitor. However, if the patient moves or if the patient's spine flexes during surgery the electronic orientation monitor must be docked with the brace and recalibrated, and if this patient movement is not noticed by the surgeon then the prosthetic component may be misaligned.

International Publication No. WO2004/112610 describes a device for detecting and measuring a change in angular position with respect to a reference plane, and its use in surgical procedures for orienting various instruments, prostheses and implants with respect to anatomical landmarks. In total hip replacement surgery, for example, the device may be configured by connecting an alignment guide to the device, and placing the guide in contact with the acetabular rim of the acetabulum before “zeroing” the device. Again, if there is any movement in the patient's acetabulum after the device is calibrated, the device must be recalibrated for correct placement of the acetabular cup. If this movement is not noticed by the surgeon, then the acetabular cup may be misaligned.

A difficulty with the system and device discussed in the preceding paragraphs is that when a surgeon is performing an operation, it is often very important that the operation is safely concluded in the shortest possible length of time, and it can take time to ensure that the system and device remain correctly calibrated in use. One very important reason for this is that hip replacement procedures are often performed on elderly or infirm patients who need to be under an anaesthetic for the duration of the operation. Another reason is that operations which can be performed in a shorter period of time utilise fewer hospital resources such as surgeons, operating theatres and anaesthetists.

A further difficulty with the system and device discussed in the preceding paragraphs, and with total hip replacement operations in general, is that when surgery is performed in the side-lying position (the most common position utilised for total hip replacement surgery) it is extremely important to control pelvic position and ensure stability of the pelvis, as the leg is manipulated during surgery. However, this is difficult to achieve and contributes to the variability in acetabular cup positioning.

To illustrate the scale of this problem, it has been estimated that 959,000 primary and revision total hip replacement procedures were undertaken per year in the United States, Spain, Portugal, The Netherlands, Canada, France, Italy Switzerland and Germany (Kurtz, S. M. (2010) Paper #365. Presented at the 56th Annual Meeting of the Orthopaedic Research Society. Mar. 6-9, 2010. New Orleans). The number of procedures is only expected to increase as an increasingly aging population is driving growth in the orthopaedics market worldwide. Furthermore, hip prostheses at present last on average 12 to 15 years even though manufacturers' laboratory test conditions indicate they should last 30 years. In the United States alone, it has been estimated that the cost of revision surgery for total hip replacement procedures exceeds US$1 billion per annum (Katz, J. N. (2007) The Orthopaedic Journal at Harvard Medical School 9:101-106).

The present invention is directed to, inter alia, a surgical system and to methods of using the surgical system, which may at least partially overcome at least one of the abovementioned disadvantages or provide the consumer with a useful or commercial choice.

### SUMMARY OF INVENTION

Embodiments of the present invention provide a system for more accurate placement and/or orientation of a surgical device, such as a prosthetic component or surgical instrument, especially an acetabular cup or a reamer.

In a first aspect, the present invention provides a surgical system for monitoring the orientation of a surgical device relative to a patient's anatomy, the system comprising:
a. A patient sensor for sensing the orientation of the patient's anatomy;
b. An orientation sensor for sensing the orientation of a surgical device; and
c. A monitor for monitoring the orientation of the surgical device relative to the sensed patient's anatomy.

Advantageously, the surgical system may allow dynamic monitoring of the orientation of a surgical device relative to a patient's anatomy. This means that any patient movement during an operation is sensed by the patient sensor, and the sensed orientation of the surgical device is updated in view of this movement. Consequently, the surgical system may allow, for example, a prosthetic component such as an acetabular cup to be placed and/or oriented more accurately.

The system may be used in orthopaedic surgical procedures. The system may be for surgery on the joint of a patient. In one embodiment, the system is used in joint surgery, especially in joint reconstruction, and most especially in total joint replacement. In one embodiment, the joint is selected from the group consisting of a hip, a shoulder, a knee, an ankle, a finger, a thumb, a toe (especially the first metatarsophalangeal (MTP) joint or the big toe), an elbow, and a wrist; especially a hip or a shoulder; most especially a hip. The surgical system may be for use in, for example, total hip replacement surgery, total knee arthroplasty, high tibial osteotomy, total shoulder replacement surgery, total wrist replacement surgery, total ankle replacement surgery, surgery to orient the thumb, fingers or toes or total elbow replacement surgery. The surgical system may be especially for use in total hip replacement surgery or in total shoulder replacement surgery; most especially in total hip replacement surgery. In a further embodiment, the surgical system may be for use in orthopaedic resurfacing, such as in hip resurfacing.

The patient's anatomy that is sensed by the patient sensor may vary depending on the surgical procedure. In surgery on the hip, for example, the orientation of the patient's pelvis may be sensed. In surgery on the shoulder, the patient's anatomy that is sensed may be the scapula. In surgery on the knee, the patient's anatomy that is sensed may be the femur or the tibia, especially the femur. In surgery to orient the first metatarsophalangeal (MTP) joint, the patient's anatomy that is sensed may be the medial malleolus. In surgery on the wrist the patient's anatomy that is sensed may be the radius or ulna. In surgery on the elbow the patient's anatomy that is sensed may be the humerus, the radius or the ulna, especially the humerus. In surgery on the ankle, the patient's anatomy that is sensed may be the tibia or fibula. Therefore, the patient's anatomy may be selected from the group consisting of the pelvis, the scapula, the femur, the tibia, the medial malleolus, the radius, the ulna, the humerus, the tibia and the fibula; especially the pelvis or the scapula; most especially the pelvis.

The patient sensor may be placed in connection with or remote to the patient's anatomy, provided that the orientation of the patient's anatomy may be sensed. In one embodiment, the patient sensor is mountable relative to a patient's anatomy, and the patient sensor is especially mountable to a patient's anatomy. Therefore, in surgery on the hip, the patient sensor may be mountable relative to, and especially mountable to, the patient's pelvis. The patient sensor may be placed in any suitable position on the patient's pelvis. For example, in a hip replacement procedure the patient sensor may be mountable relative to, especially mountable to, the patient's iliac crest or sacrum, especially the sacrum. In other surgical procedures, the patient sensor may be mountable relative to, and especially mountable to, a scapula, a femur, a medial malleolus, a tibia, a radius, an ulna, a humerus, or a fibula; especially a scapula.

The patient sensor may be mountable relative to the patient in any suitable way. The patient sensor may comprise at least one fastener for mounting the sensor relative to a patient's anatomy. For example, the fastener may be an adhesive, especially an adhesive layer, for adhering the sensor to the patient's skin. While any suitable type of adhesive may be used, the adhesive may be especially a similar type to those used on the electrodes when performing electrocardiography (ECG). The fastener may also be strapping for holding the sensor against the patient's anatomy. The fastener may also be adhesive tape, such as Elastoplast®, for taping the patient sensor to the patient's anatomy. The fastener may be pedicle screws for screwing the sensor onto a patient's bone, such as the pelvis. The fastener may also be suction caps to secure the patient sensor against, or relative to, the patient's anatomy. The fastener may also comprise a hook and loop fastener, especially a circular hook and loop fastener. An example hook and loop fastener is Velcro™.

Combinations of two or more of the above fasteners may be used. For example, the patient sensor may comprise an adhesive layer, for adhering the sensor to the patient's skin, and adhesive tape, such as Elastoplast®, may be used to further tape the patient sensor to the patient's anatomy.

The fasteners may be located at two or more positions on the patient sensor; especially at between two and six positions; more especially at between three and five positions; most especially at four or five positions. For example, the patient sensor may comprise two or more separately located adhesive layers; especially between three and six separately located adhesive layers; most especially four or five separately located adhesive layers.

The patient sensor may be substantially rigid, or it may be configured to allow mounting of the sensor to different sites on a patient's anatomy. In one embodiment, the patient sensor is mouldable to a patient's anatomy, or flexible so as to conform to a patient's anatomy.

In one embodiment, the orientation sensed by the patient sensor is one or more of pitch, roll and yaw; especially two or more of pitch, roll and yaw. In a preferred embodiment, the patient sensor is for sensing the pitch, roll and yaw of the patient's anatomy. Pitch and roll are sensed relative to the horizontal, and yaw is sensed relative to the patient axial line as determined by the surgeon. In hip surgery, for example, yaw may be sensed relative to the patient sagittal plane, which is the longitudinal planar axis, passing through the head, spine and pelvis of the body, bisecting the body into two equal parts.

The patient sensor may comprise at least one sensor for sensing the orientation of the patient's anatomy. The at least one sensor senses at least one of pitch, roll and yaw, especially at least two of pitch, roll and yaw, and most especially senses the pitch, roll and yaw of the patient's anatomy. In one embodiment, the at least one sensor is selected from one or more of a gyroscope, a magnetometer, an accelerometer, an inclinometer and an inertial sensor. Exemplary sensors/sensor combinations are available from Xsens (http://www.xsens.com). In one embodiment, the sensor may act in combination with laser pointing, or a beacon located in proximity to the sensor (for example in the operating theatre), and this embodiment may be especially advantageous when sensing yaw.

The patient sensor may be connected to a power supply. For example, in use the patient sensor may be connected to an external power supply such as a wall socket. Alternatively, the patient sensor may comprise a power supply, such as a battery. Any suitable battery may be used. The battery may be rechargeable. For example, the battery may be rechargeable by coupling the sensor to a power cord or by inductive charging. A battery rechargeable by inductive charging may be especially advantageous if the patient sensor is sterilisable. Alternatively, the battery may be sterilisable and/or replaceable, in which case the battery may be rechargeable or not rechargeable. In a further embodiment, the patient sensor may be disposable in which case the battery may not be replaceable. The power supply may be, for example, a sterilisable lithium ion battery. One, two or more of such batteries may be used. The patient sensor may also comprise an on/off switch for activating the sensor.

The patient sensor may comprise a sensing portion and a base. The sensing portion may be mountable to the base in any suitable way, such as by friction-fit, interference-fit, tongue in groove, bayonet coupling, via a hook and loop fastener (such as Velcro™) or the like. In one embodiment, the base of the patient sensor may comprise a docking port for mounting the sensing portion. The sensing portion may slideably engage with the docking port, or the sensing portion may be screwed into the docking port.

The sensing portion may comprise the at least one sensor for sensing the orientation of the patient's anatomy, the power supply and/or switch, and the base may be mountable relative to, and especially mountable to, the patient's anatomy. The patient sensor, especially the sensing portion, may also comprise an indicator, such as a light, for indicating the power remaining in the power supply.

The base may comprise a fastener (as discussed above), such as an adhesive or pedicle screws. In one embodiment, the base comprises a body and at least one coupler, wherein the body is mountable to the at least one coupler, and the at least one coupler may be mountable relative to the patient's anatomy. In an exemplary embodiment, the at least one coupler comprises a fastener (as discussed above), especially an adhesive layer. The coupler may also comprise a clamp for clamping the coupler to the body of the patient sensor base. Any suitable clamp may be used. One exemplary arrangement is that the body of the patient sensor base defines at least one aperture (preferably between two and six apertures; more preferably between three and five apertures; most preferably four or five apertures), each aperture for accommodating at least one coupler. The coupler may comprise, for example, an adhesive layer and a clamp which comprises a threaded rod, a securing collar and a nut.

The sensing portion may be flexible or substantially rigid, especially substantially rigid. The base may also be substantially rigid or flexible to conform to the patient's anatomy. In one embodiment, the base is mouldable to the patient's anatomy.

The patient sensor and/or the sensing portion may comprise a housing. For example, the sensing portion housing may encase the at least one sensor and/or the power supply. The housing and/or the base may be made of metal or plastic, and may include a lid which may be opened by rotation about a hinge. A releasable fastener may secure the housing closed.

The patient sensor, sensing portion and/or the base may be made of any suitable material. For example, the body of the patient sensor base may be made of a plastic such as polytetrafluoroethylene (PTFE). The base is especially made of an X-ray translucent material. The patient sensor, sensing portion and/or the base may be of any suitable shape, and the most appropriate shape will vary depending on, for example, the portion of the patient's anatomy the patient sensor is to be mounted relative to, and the size and shape of the patient at this anatomy.

The patient sensor, sensing portion and/or the base may be disposable or sterilisable. If the patient sensor or the sensing portion is sterilisable, then the patient sensor or the sensing portion may comprise an insulator for insulating the electronic components from chemical, thermal or pressure effects. The insulator may protect these components against standard autoclave sterilising or gas approved sterilisation. In one embodiment, the insulator is a casing. In one embodiment, all components of the patient sensor, with the possible exceptions of the at least one sensor and the power supply, may be sterilisable. However, non-sterile components, such as an at least one sensor and power supply, may be used in the system if these components are inserted inside a sterile housing.

The surgical system may comprise at least one patient sensor. In one embodiment, the surgical system comprises two patient sensors, and the surgical system especially consists of two patient sensors. Using more than one patient sensor may be advantageous. For example, if only one patient sensor is used the calibration of the system may be impaired if the sensor is bumped or knocked from the patient's anatomy during operation. If the surgical system comprises more than one patient sensor, then the patient sensors may be identical to each other or different.

The system also comprises an orientation sensor for sensing the orientation of a surgical device. The surgical device used depends upon the surgical operation being performed. In one embodiment, the surgical device is a surgical implement. The surgical implement may be, for example, a reamer. In hip replacement surgery, the reamer may be an acetabular reamer.

The surgical device may also be a prosthetic component. In one embodiment, the prosthetic component is a prosthetic component for hip replacement surgery, especially an acetabular cup. The acetabular cup may be for receiving a femur or a prosthetic femur.

Different surgical devices may be used for different operations. For example, in shoulder replacement surgery the surgical device may be a glenoid cavity reamer. Alternatively the surgical device may be a humeral cup. The humeral cup may be for receiving a humerus or a prosthetic humerus. In another embodiment, the surgical device may be a jig, especially for operating on a knee. In this embodiment, the patient sensor may be mountable relative to a femur, and the orientation sensor mounted relative to a jig. The jig may be oriented on the knee before a surgeon excises bone so that a prosthetic knee may be optimally fitted. Advantageously, by using the surgical system in this operation potential problems due to poor orientation may be minimised, such as “bandy” legs.

In another embodiment, the surgical system further comprises a surgical device for surgical use on the patient's anatomy. The surgical device may be as discussed above. In one example, the surgical device comprises the orientation sensor, especially such that the orientation sensor is not removable (in this example the surgical device is especially an acetabular reamer). In another example, the orientation sensor forms part of the surgical device, for example such that a component of a surgical device (such as an acetabular reamer driver, or a portion of an acetabular reamer between the cutting blade and the motor) is substituted with the orientation sensor. Similarly, when the surgical device is a prosthetic component, a placement device for placing the prosthetic component may comprise the orientation sensor.

The orientation sensor may be placed in connection with or remote to the surgical device, provided that the orientation of the surgical device is sensed. In one embodiment, the orientation sensor may be mountable relative to a surgical device. For example, the orientation sensor may be mounted to an instrument or to a guide which is mountable to the surgical device. When the surgical device is a prosthetic component, the orientation sensor is especially mountable relative to the surgical device. For example, the orientation sensor may be mountable to a placement device for placing a prosthetic component. The prosthetic component may be releasably attached to the placement device. In one embodiment, the orientation sensor is mountable to a reamer or mountable to a placement device for placing a prosthetic component.

In another example, the orientation sensor is mountable to a surgical device. The orientation sensor may be mountable to the surgical device in any suitable way. For example, the orientation sensor may be mountable in a docking port on the surgical device or in a docking port on an instrument which is mountable to the surgical device. The docking port may be adjustable to accommodate an orientation sensor in various orientations. Furthermore, the docking port may be lockable. The docking port may be oriented to a specific angle and be adjustably inclined to the axial line. Preferably, the docking port may be lockable by deforming a portion of the port (which may be advantageous for sterilisation). For example, the docking port may comprise a flexible plastic hinge between the docking port door and the remainder of the port. The docking port door may be closed by bending the flexible plastic. In a further embodiment, the orientation sensor may comprise a fastener for fastening the orientation sensor to a reamer or placement device.

In a further embodiment, the orientation sensor comprises a sensing portion and a mounting, wherein the sensing portion is mountable to the mounting. The mounting may be for mounting the orientation sensor to a reamer or to a placement device. The sensing portion may be mountable to the mounting in any suitable way, such as by friction-fit, interference-fit, tongue in groove, bayonet coupling, via a hook and loop fastener (such as Velcro™) or the like. In one embodiment, the mounting of the orientation sensor may comprise a docking port for mounting the sensing portion. The sensing portion may slideably engage with the docking port, or the sensing portion may be screwed into the docking port.

It would be appreciated that any suitable design for the mounting may be used, and the design of the mounting will vary depending upon the reamer or placement device to which the orientation sensor is mountable. The mounting may include one or more of a docking port (such as discussed in the preceding paragraph) for mounting the sensing portion, a shock absorber (especially for absorbing shocks associated with, for example, driving a prosthetic component into the required position), a clamp for clamping the orientation sensor to a reamer or placement device, and a spacer for spacing the docking port from the clamp. The clamp may include a fastener. Furthermore, the sensing portion may slideably engage with the docking port, or the sensing portion may be screwed into the docking port. An advantage of using a spacer is that when the system is used, the orientation sensor is offset and is less likely to obscure a surgeon's line of sight down the central axis of the instrument being used (e.g. an acetabular reamer or acetabular cup placement device). In one embodiment, the orientation sensor sensing portion is offset from the longitudinal axis of the surgical device (or the longitudinal axis of a placement device, for example).

In another embodiment, the mounting may form part of the surgical device, or an instrument or guide which is mountable to the surgical device. For example, if the surgical device is an acetabular reamer, the mounting may form part of the reamer (e.g. a component of the reamer may be substituted with the mounting, and this component may be an acetabular reamer driver, or a portion of the reamer between the cutting blade and the motor). For the avoidance of doubt, the phrase “mountable relative to a surgical device” includes circumstances in which, for example, the mounting of the orientation sensor forms part of the surgical device or part of a placement device for placing a prosthetic component. Similarly, the phrase “mountable to a reamer” includes circumstances in which, for example, the mounting of the orientation sensor forms part of the reamer.

Any suitable shock absorber may be used, and the shock absorber may absorb shocks in one, two or more directions, especially in one or two directions. The shock absorber may, for example, comprise a biasing member (such as a spring), and/or comprise pneumatic absorption (for example, like a piston). Similarly, any suitable fastener may be used, including bolts and lock nuts. It may not be necessary for the orientation sensor to include a shock absorber when the orientation sensor is attached to an acetabular reamer, as the effect of the acceleration of the acetabular reamer on the orientation sensor may be effectively ameliorated electronically.

In a further embodiment, the mounting may include a cradle or bracket for holding a monitor and/or a communicator (as discussed further below). Such a cradle or bracket may be designed to absorb shock, for example similar to those designed for motorcycle GPS.

In one embodiment, the orientation sensed by the orientation sensor is one or more of pitch, roll and yaw; especially two or more of pitch, roll and yaw. In a preferred embodiment, the orientation sensor is for sensing the pitch, roll and yaw of the surgical device. The orientation sensed by the orientation sensor may be the same as the orientation sensed by the patient sensor (i.e. both the patient sensor and the orientation sensor may sense pitch and roll, especially pitch, roll and yaw).

The orientation sensor may comprise at least one sensor for sensing the orientation of the surgical device. The at least one sensor may be as described for the patient sensor, but it need not be the same.

The orientation sensor may be connected to a power supply, or the orientation sensor may especially comprise a power supply, such as a battery. These components may be as discussed for the patient sensor. In a further embodiment, the orientation sensor may be disposable in which case the battery may not be replaceable. The orientation sensor may also comprise an on/off switch for activating the sensor. In a further embodiment, the orientation sensor may be powered by the surgical device (such as when the surgical device is an acetabular reamer).

The sensing portion of the orientation sensor may comprise the at least one sensor for sensing the orientation of the surgical device, the power supply and/or switch. The orientation sensor, especially the sensing portion, may also comprise an indicator, such as a light, for indicating the power remaining in the power supply.

The orientation sensor and/or the sensing portion may comprise a housing. For example, the sensing portion housing may encase the at least one sensor and/or the power supply. The housing may be made of metal or plastic, and may include a lid which may be opened by rotation about a hinge. A releasable fastener may secure the housing closed.

The orientation sensor, and components of the orientation sensor, may be made of any suitable material. For example, the orientation sensor mounting may be made from a sterilisable material, such as metal or plastic, especially metal, more especially stainless steel. The mounting may be made of a non-magnetisable material. The orientation sensor may be of any suitable shape and may be flexible or substantially rigid.

The orientation sensor, sensing portion and/or the mounting may be disposable or sterilisable. If the orientation sensor or the sensing portion is sterilisable, then the orientation sensor or the sensing portion may comprise an insulator for insulating the electronic components from chemical, thermal or pressure effects. The insulator may protect these components against standard autoclave sterilising or gas approved sterilisation. In one embodiment, the insulator is a casing. In one embodiment, all components of the orientation sensor, with the possible exceptions of the at least one sensor and the power supply, may be sterilisable. However, non-sterile components, such as the at least one sensor and power supply, may be used in the system if these components are inserted inside a sterile housing.

The surgical system further comprises a monitor for monitoring the orientation of the surgical device relative to the sensed patient's anatomy. The monitoring occurs in real time. The monitor may receive the sensed orientation of the patient's anatomy and the sensed orientation of the surgical device, and then monitor the orientation of the surgical device relative to the sensed patient's anatomy. The monitor may monitor at least one of (and especially all of) the pitch, roll and yaw of the surgical device relative to the patient's anatomy.

The monitor may store or data log information from the surgery. This information may be simply a log which indicates how many operations have been performed using the system. The information may also be a full or partial record of the sensed orientation of the patient sensor (or the patient's anatomy), the sensed orientation of the orientation sensor (or the surgical device) and/or the orientation of the orientation sensor relative to the patient sensor (or the surgical device relative to the patient's anatomy). The information may be transferrable from the monitor to another medium, such as to a memory card. In one embodiment, the monitor comprises a data processor, a computer-readable storage medium, a microprocessor and/or a central processing unit (CPU). The monitor need not be a single piece of equipment.

The surgical system may further comprise a communicator for communicating (especially to a surgeon) one or more of, especially all of, the sensed orientation of the patient's anatomy, the sensed orientation of the surgical device and the monitored orientation of the surgical device relative to the sensed patient's anatomy. The communicator may visually or audibly communicate. For example, the communicator may visually communicate the sensed orientation of the patient's anatomy, the sensed orientation of the surgical device and the monitored orientation of the surgical device relative to the sensed patient's anatomy via a visual display or via an audio speaker, especially via a visual display.

For example, the communicator may communicate with the surgeon visually, such as on a computer screen. This may involve graphically showing the monitored orientation of the surgical device relative to the sensed patient's anatomy. Alternatively, the communicator may numerically show the yaw, roll and pitch of the sensed surgical device relative to the sensed patient's anatomy.

The patient sensor or the orientation sensor may be co-located with the monitor and/or the communicator. In one embodiment, the monitor, communicator and orientation sensor are co-located. In another embodiment, the monitor, communicator and patient sensor are co-located. For example, the communicator or the monitor and communicator may be holdable in a cradle or bracket in the orientation sensor mounting (as discussed above), or in the orientation sensor docking port. In this example, the communicator or the monitor and communicator may be encased in use within a sterilisable housing which is held in, or forms part of, the cradle, bracket or docking port. An advantage of co-locating the patient sensor or the orientation sensor with the monitor and/or the communicator is that the surgeon may advantageously be able to better see a read-out from the communicator during the operation (especially if the communicator graphically shows the orientation of the surgical device relative to the sensed patient's anatomy).

In a further embodiment, a single device may include the monitor, communicator and/or sensing portion of the patient sensor. In this embodiment, the device including the monitor/communicator/patient sensor sensing portion may be mountable to the patient sensor base. In another embodiment, a single device may include the monitor, communicator and/or sensing portion of the orientation sensor. In this embodiment, the monitor/communicator/orientation sensing portion may be mountable to the orientation sensor mounting.

In other embodiments, the patient sensor comprises the monitor or the communicator, or both the monitor and the communicator. This may be advantageous as the patient sensor, if positioned on the patient's iliac crest, may be in the surgeon's line of sight when orienting the surgical device. In further embodiments, the orientation sensor comprises the monitor or the communicator, or both the monitor and the communicator.

Alternatively, the monitor or communicator (especially both the monitor and the communicator) may be remote to the orientation sensor and the patient sensor. An advantage of a remote monitor and communicator is that the monitor and communicator may not require sterilisation between operations. Sterilisation conditions could potentially damage some of the electronic components in the monitor and communicator. In alternative embodiments, the monitor and/or the communicator are sterilisable or disposable.

The monitor and communicator may be co-located, for example, in a computer (including devices such as laptops, tablets, smartphones, PDAs, iPads®, iPhones® and iPods®).

The surgical system may comprise at least one communicator. For example, the surgical system may comprise two, three or four communicators. In an exemplary embodiment, the surgical system may comprise a first communicator that is located at, or adjacent to the orientation or patient sensor, and a second communicator at another position in the operating theatre (for example on a wall). Where there are a plurality of communicators, the information displayed on each communicator may be the same or different. For example, one communicator may provide a graphical display of the orientation of the pitch, roll and yaw of the surgical device relative to the sensed patient's anatomy, and a second communicator may provide a numerical display of the same information.

The patient sensor, orientation sensor, monitor and optionally the communicator are in communication. In one embodiment, the patient sensor is in communication with the monitor, and the orientation sensor is in communication with the monitor. The patient sensor and orientation sensor may be in one or two way communication with the monitor, especially two-way communication. The patient sensor, orientation sensor, monitor and communicator may also be in communication in any other suitable way. For example, the patient sensor may be in communication with the orientation sensor, which in turn is in communication with the monitor. The monitor in turn may be in communication with the communicator.

This communication may be via an electrical cable or it may be wireless, especially wireless. The communication should be non-invasive so as to avoid interfering with other electronic equipment in the operating theatre. For example, the communication may be via a wireless protocol for exchanging data over a short distance personal area network. An example of such a wireless protocol is Bluetooth™ or Wi-Fi. The communication may be at a frequency and bandwidth that does not interfere with other hospital equipment. Furthermore, the wattage of the components may be configured to be allowable in operating theatres.

In another embodiment, the patient sensor, orientation sensor, monitor and/or the communicator are not sterilisable or disposable. In this embodiment, the system may comprise one or more sterile containers into which these components may be placed prior to use. In a further embodiment, a portion of the patient sensor, orientation sensor, monitor and/or the communicator may be sterilisable, and unsterilized portions of these components may be inserted into the sterile portion prior to use. For example, the housing of the orientation sensor and/or the patient sensor may be sterilisable, and the at least one unsterilized sensor, battery and/or electronic components may be placed inside the sterile housing, prior to use.

In another embodiment, the surgical system further comprises a cradle for calibrating the orientation and patient sensors. The cradle may accommodate the orientation and patient sensors, or the sensing portions of the orientation and patient sensors, especially such that the orientation and patient sensors are parallel to each other. The orientation and patient sensors may be calibrated when in the cradle, such that the pitch, roll and/or yaw for both sensors are set to zero.

In a second aspect, the present invention provides a surgical system for orienting a surgical device relative to a patient's anatomy, the system comprising:
a. A patient sensor for sensing the orientation of the patient's anatomy;
b. An orientation sensor for sensing the orientation of a surgical device;
c. A monitor for monitoring the orientation of the surgical device relative to the sensed patient's anatomy; and
d. A communicator for guiding the monitored surgical device to an optimal surgical device orientation relative to the patient's anatomy.

Features of the surgical system of the second aspect of the present invention may be as described above for the first aspect. However in this aspect, the communicator is for guiding the monitored surgical device to an optimal surgical device orientation relative to the patient's anatomy.

In this aspect, the monitor or communicator (preferably the monitor) may determine an optimal orientation of the surgical device relative to the patient's anatomy. In one embodiment, the monitor determines the optimal orientation of the surgical device by considering natural variations in the patient's anatomy. For example, many patients have a natural tilt to their pelvis, as measured by the pelvic tilt angle. The patient's pelvic tilt angle is the difference between the vertical and a line drawn between the centre of rotation of each of the patient's hips, when the patient is positioned on an operating table with one hip vertically above the other, i.e. lying on their side. The patient's pelvic tilt angle therefore may be measured with respect to roll and pitch when in this position, i.e., the forward or backward movement from the fully vertical position. The pelvic tilt angle may also be measured with respect to the degrees of inclination and anteversion of the acetabulum (the angle that the acetabulum projects from the pelvis). This angle may be determined by taking an X-ray image of the patient's pelvis prior to the operation. Thus, in one embodiment the monitor determines the optimal orientation of the surgical device by considering the patient's pelvic tilt angle. Determining the optimal orientation of the surgical device may also involve consideration of differences in one or more of the pitch, roll and yaw (especially two or more of pitch, roll and yaw; most especially all of pitch, roll and yaw) due to the position of the patient on the operating table.

Alternatively, the optimal orientation of the surgical device relative to the patient's anatomy may be provided to the monitor or the communicator, especially to the monitor.

The optimal orientation may comprise at least one of (and especially all of) the pitch, roll and yaw of the surgical device relative to the patient's anatomy.

The monitor or communicator (preferably the monitor) may also determine the difference between the optimal orientation of the surgical device and the orientation of the surgical device relative to the sensed patient's anatomy. The communicator may guide the monitored surgical device by communicating this difference to the surgeon. Therefore, the communicator may communicate (especially to a surgeon) the difference between the optimal orientation of the surgical device and the orientation of the surgical device relative to the sensed patient's anatomy. Alternatively, the communicator may indicate the orientation of the monitored surgical device and the optimal orientation of the device, allowing the surgeon to assess their relationship.

The communicator may communicate (especially to the surgeon) one or more of, especially all of, the sensed orientation of the patient's anatomy, the sensed orientation of the surgical device, the monitored orientation of the surgical device relative to the sensed patient's anatomy, the optimal orientation of the surgical device, and the orientation of the monitored surgical device relative to the optimal orientation; especially the monitored orientation of the surgical device relative to the sensed patient's anatomy, the optimal orientation of the surgical device, and the orientation of the monitored surgical device relative to the optimal orientation. In one embodiment, the communicator communicates the orientation of the monitored surgical device relative to the optimal orientation. As described above, the communicator may communicate visually or audibly, especially with the surgeon. For example, the communicator may communicate via a visual display. Alternatively, the communicator may audibly communicate, especially with the surgeon. For example, the difference between the optimal orientation of the surgical device and the monitored orientation of the surgical device may be communicated to the surgeon by one or more audio signals (where, for example, the pitch, tone or duration of the audio signal guides the surgeon).

For example, the communicator may visually communicate (especially with the surgeon) by graphically illustrating the orientation of the surgical device relative to the sensed patient's anatomy, and the optimal orientation of the surgical device. The surgeon is then able to observe the difference between these and guide the surgical device into the correct orientation. In an exemplary embodiment, the graphical display may include a horizontal and a vertical axis which bisect, with roll on one axis and pitch on the other axis, wherein the point at which the axes cross is the optimal pitch and roll for the surgical device relative to the patient's anatomy. The graphical display may also include a third axis which illustrates yaw, wherein the centre of the axis is the optimal yaw for the surgical device relative to the patient's anatomy.

In another example, the communicator may audibly communicate (especially with the surgeon). In this example, the communicator may make a beeping noise when the device is in the incorrect orientation, and a pinging noise when the device is correctly oriented. The communicator may also communicate both visually and audibly with the surgeon.

Advantageously, this aspect of the present invention may allow a surgeon to be guided during reaming of the acetabulum. Many hip replacement surgeries are unsuccessful due to misalignment of the acetabular cup. While several aligning devices have been developed to improve the accuracy of orienting the acetabular cup, it is believed that surgeons ream the acetabulum by eye. The surgical system of this aspect of the present invention may allow guided reaming of the acetabulum, to thereby minimise removal of pelvic bone and provide an improved foundation for placement and orientation of the acetabular cup. Advantageously, when the reaming of the acetabulum is guided, a rim may be created on the pelvic bone which may assist in placing and orienting the acetabular cup. Guided reaming may allow two machined surfaces (the bone and the acetabular cup) to come into contact which may provide a superior fit. If the reaming step is performed incorrectly the acetabular cup can disengage from the acetabulum, or for example, one of the patient's legs may become shorter than the other. If the reaming operation is performed incorrectly, then it may be difficult to correctly orient the acetabular cup.

Therefore in a third aspect, the present invention provides a surgical system for guided reaming of a patient's anatomy, the system comprising an orientation sensor for sensing the orientation of a reamer relative to a patient's anatomy. This system may or may not comprise a patient sensor or a monitor. However, in one embodiment the system comprises a patient sensor for sensing the orientation of the patient's anatomy, an orientation sensor for sensing the orientation of a reamer, and/or a monitor for monitoring the orientation of the reamer relative to the sensed patient's anatomy. The patient's anatomy is especially the pelvis and the reamer is especially an acetabular reamer. The surgical system may further comprise a communicator for guiding the monitored reamer to an optimal reamer orientation relative to the patient's anatomy. The surgical system may also further comprise a reamer. Features of the surgical system of the third aspect of the present invention may be as described above for the first or second aspect.

In a fourth aspect, the present invention provides a placement device for surgical placement of a prosthetic component, wherein the device comprises a driver capable of generating at least one impulse to drive the prosthetic component into a patient's anatomy.

The device of this aspect of the present invention may allow more accurate placement of an acetabular cup over current techniques such as using a hammer to drive the cup into the acetabulum.

In one embodiment, the driver is capable of generating two or more successive impulses to drive the prosthetic component into a patient's anatomy. The driver may be capable of generating a pulsing action, especially an axial pulsing action. The timing of the impulses may be adjustable.

In a further embodiment, the driver is capable of detecting when the prosthetic component cannot be driven further into the patient's anatomy. This may be achieved by detecting the movement of the prosthetic component relative to the device following an impulse.

The impulse may be generated by air pressure, ultrasound, or by a striker for impacting the prosthetic component, especially by a striker for impacting the prosthetic component. In one example, the striker may operate by compressing a spring with a power operated cam. The impulse may be generated by one or more cams, especially two cams. In one example, the impulse may be generated by contra rotating cams. In another example, the impulse may be generated by piezoelectric action. In one embodiment, the impulse is generated pneumatically, hydraulically or by a motor, and preferably the impulse is generated pneumatically.

The prosthetic component may be as described elsewhere in this specification, and may be especially a humeral or acetabular cup, most especially an acetabular cup.

In one embodiment, the placement device further comprises a mounting for mounting the prosthetic component relative to the driver. The mounting may be capable of attaching or releasing a prosthetic component in one or two steps. For example, the mounting may comprise a spring loaded coupling. The mounting may be for mounting an instrument which is coupled to the prosthetic component. For example, the mounting may be for mounting a shaft which is coupled to an acetabular cup. In one embodiment, the mounting is capable of coupling to a plurality of different length shafts, each with a different thread to suit the various acetabular cups used by a surgeon.

In one embodiment, the driver and optionally the mounting of the placement device are detachable. In this embodiment, the driver and the mounting may be replaceable with a reamer. For example, the reamer output drive shaft and driver/mounting may be arranged for snap connection, especially a connection compatible with existing devices for surgery. In this embodiment, the speed of the reamer may be adjustable, and/or the frequency of the impulses of the driver may be adjustable. In this embodiment, the device may be used for reaming and for placement of the prosthetic component.

An orientation sensor for sensing the orientation of the prosthetic component, as described elsewhere in the specification, may be mountable relative to the device. For example, the orientation sensor may be mounted to an instrument or to a guide which is mountable to the placement device. In another example, the orientation sensor is mountable to the placement device.

The orientation sensor may be mountable relative to the device in any suitable way. For example, the orientation sensor (or the sensing portion of the orientation sensor) may be mountable in a docking port on the device or in a docking port on an instrument which is mountable to the surgical device. The docking port may be adjustable to accommodate an orientation sensor (or sensing portion of an orientation sensor) in various orientations. Furthermore, the docking port may be lockable. The docking port may be oriented to a specific angle and be adjustably inclined to the axial line. Preferably, the docking port may be lockable by deforming a portion of the port (which may be advantageous for sterilisation). For example, the docking port may comprise a flexible plastic hinge between the docking port door and the remainder of the port. The docking port door may be closed by bending the flexible plastic. The docking port may be set at an appropriate angle (nominally 45°) and adjustable inclination to the axial line. The orientation sensor sensing portion may slideably engage with the docking port, or the sensing portion may be screwed into the docking port. Features of the orientation sensor sensing portion may be as described elsewhere in this specification.

In a fifth aspect, the present invention provides a surgical system comprising the placement device of the fourth aspect of the present invention. Features of the surgical system of the fifth aspect of the present invention may be as described in the first or second aspects of the present invention.

In one embodiment, the system comprises a patient sensor for sensing the orientation of the patient's anatomy, an orientation sensor for sensing the orientation of the prosthetic component, and a monitor for monitoring the orientation of the prosthetic component relative to the sensed patient's anatomy. The patient's anatomy is especially the pelvis. The surgical system may further comprise a communicator for guiding the monitored prosthetic component to an optimal placement orientation relative to the patient's anatomy.

In a sixth aspect, the present invention provides a patient sensor for sensing the orientation of a patient's anatomy, wherein the patient sensor is for communicating the orientation of the sensed patient's anatomy to a monitor. In one embodiment, the monitor is for monitoring the orientation of a surgical device relative to the sensed patient's anatomy.

In a seventh aspect, the present invention provides a patient sensor for sensing the orientation of a patient's anatomy, wherein the patient sensor is for monitoring the sensed orientation of a surgical device relative to the sensed patient's anatomy.

In an eighth aspect, the present invention provides an orientation sensor for sensing the orientation of a surgical device, wherein the orientation sensor is for communicating the orientation of the surgical device to a monitor. In one embodiment, the monitor is for monitoring the orientation of the surgical device relative to a sensed patient's anatomy.

In a ninth aspect, the present invention provides an orientation sensor for sensing the orientation of a surgical device, wherein the orientation sensor is for monitoring the orientation of a surgical device relative to a sensed patient's anatomy.

In a tenth aspect, the present invention provides a monitor for monitoring the orientation of a surgical device relative to a patient's anatomy, wherein the monitor is for receiving a communicated orientation of a patient's anatomy from a patient sensor, and wherein the monitor is for receiving a communicated orientation of a surgical device from an orientation sensor.

In an eleventh aspect, the present invention provides a surgical system for sensing the orientation of a surgical device relative to a patient's anatomy, the system comprising:
a. A patient sensor for sensing the orientation of the patient's anatomy; and
b. An orientation sensor for sensing the orientation of the surgical device relative to the sensed patient's anatomy.

In a twelfth aspect, the present invention provides a surgical system for monitoring the orientation of a surgical device relative to a patient's anatomy, the system comprising:
a. A patient sensor for sensing the orientation of the patient's anatomy; and
b. A monitor for receiving a communicated orientation of a surgical device from an orientation sensor, and for monitoring the orientation of the surgical device relative to the sensed patient's anatomy.

In a thirteenth aspect, the present invention provides a surgical system for monitoring the orientation of a surgical device relative to a patient's anatomy, the system comprising:
a. An orientation sensor for sensing the orientation of a surgical device; and
b. A monitor for receiving a communicated orientation of a patient's anatomy from a patient sensor, and for monitoring the orientation of the surgical device relative to the sensed patient's anatomy.

Features of the sixth to thirteenth aspects of the present invention may be as described above for the first or second aspect.

In a fourteenth aspect, the present invention provides a method of monitoring the orientation of a surgical device relative to a patient's anatomy, the method comprising:
a. Sensing the orientation of the patient's anatomy; and
b. Monitoring the orientation of the surgical device relative to the sensed patient's anatomy.

The method may be especially performed in hip replacement surgery. Such hip replacement surgery may be performed to relieve hip pain associated with conditions such as osteoarthritis, inflammatory arthritis, bone necrosis (osteonecrosis), trauma or the degradation of the join surfacing caused by general wear and tear.

In one embodiment, the method comprises mounting a patient sensor relative to a patient's anatomy. The patient sensor, patient's anatomy, and the manner in which the patient sensor may be mounted relative to a patient's anatomy may be as discussed above in relation to the first and second aspects of the present invention. In this embodiment, the patient sensor senses the orientation of the patient's anatomy.

In another embodiment, the method comprises providing a surgical device for surgical use on the patient's anatomy. The surgical device may be as discussed in relation to the first and second aspects of the present invention.

In a further embodiment, the method comprises mounting an orientation sensor relative to a surgical device. The orientation sensor, surgical device and the manner in which the orientation sensor may be mounted to the surgical device may be as discussed above in relation to the first and second aspects of the present invention. In this embodiment, the orientation sensor senses the orientation of the surgical device.

Therefore, in one embodiment the present invention provides a method of monitoring the orientation of a surgical device relative to a patient's anatomy, the method comprising:
a. Mounting a patient sensor relative to a patient's anatomy;
b. Sensing the orientation of the patient's anatomy with the patient sensor;
c. Mounting an orientation sensor relative to a surgical device;
d. Sensing the orientation of the surgical device with the orientation sensor; and
e. Monitoring the orientation of the surgical device relative to the sensed patient's anatomy with a monitor.

In one embodiment, the method first comprises securing the patient to an operating table. The patient's anatomy may be especially substantially immobilised before the surgery.

Before mounting the patient sensor and the orientation sensor, it may be advantageous to place both the patient sensor and the orientation sensor on the same plane (for example, this may involve placing the sensors side by side on a table or in a cradle). In this step, the sensors may be calibrated so that they share the same orientation readings. After the patient sensor has been mounted relative to the patient's anatomy, the patient sensor may be “zeroed”, and any change in orientation readings in zeroing the sensor may be reflected in (or transferred to) the orientation sensed by the orientation sensor. The sensors may then remain activated until the surgical device has been used or inserted. Alternatively, the surgical system may be used without calibrating the sensors.

In another embodiment, the method comprises providing a monitor for monitoring the orientation of the surgical device relative to the sensed patient's anatomy. The monitor may be as discussed above in relation to the first and second aspects of the present invention.

In a further embodiment, the method comprises the step of communicating to a surgeon the monitored orientation of the surgical device relative to the patient's anatomy. A communicator may be used for this step, and the communicator may be as described in relation to the first and second aspects of the present invention.

In a further embodiment, the patient's anatomy is the pelvis, and the surgical device is an acetabular reamer. When reaming, the surgical device may move due to the force of the reaming operation. To limit this movement a device or guide may be used.

The method may also comprise using a support for the surgical device. For example, a support may be connected to an operating table by, for example, a bayonet connection to a fitting mounted on the operating table attachment rail. The support may be adjustable, for example using clamps, to allow positioning of the surgical device as required. If the surgical device is a reamer, for example, the support may be used to support and guide the reamer during the surgical operation. The support may also include a slide for guiding the surgical device.

The method of this aspect may be used for total hip replacement surgery. As discussed above, hip replacement surgery may include: (i) severing the femoral head and dislocating the head of the femur from the acetabulum; (ii) reaming the acetabulum to fit an acetabular cup; (iii) inserting the acetabular cup; (iv) shaping the femoral canal, and then fitting a prosthetic femoral component into the canal; and (v) fitting the prosthetic femoral component to the acetabular cup. The method of this aspect may be advantageously performed to monitor the orientation of the surgical device used in steps (ii) and (iii).

In a fifteenth aspect, the present invention provides a method of orienting a surgical device relative to a patient's anatomy, the method comprising:
a. Sensing the orientation of the patient's anatomy;
b. Monitoring the orientation of the surgical device relative to the sensed patient's anatomy;
c. Providing an optimal orientation of the surgical device; and
d. Guiding the monitored surgical device to the optimal orientation.

In one embodiment, the method comprises mounting a patient sensor relative to a patient's anatomy. The patient sensor, patient's anatomy, and the manner in which the patient sensor may be mounted relative to a patient's anatomy may be as discussed above in relation to the first and second aspects of the present invention. In this embodiment, the patient sensor senses the orientation of the patient's anatomy.

In another embodiment, the method comprises providing a surgical device for surgical use on the patient's anatomy. The surgical device may be as discussed in relation to the first and second aspects of the present invention.

In a further embodiment, the method comprises mounting an orientation sensor relative to a surgical device. The orientation sensor, surgical device and the manner in which the orientation sensor may be mounted relative to the surgical device may be as discussed above in relation to the first and second aspects of the present invention. In this embodiment, the orientation sensor senses the orientation of the surgical device.

Therefore, in one embodiment the present invention provides a method of monitoring the orientation of a surgical device relative to a patient's anatomy, the method comprising:
a. Mounting a patient sensor relative to a patient's anatomy;
b. Sensing the orientation of the patient's anatomy with the patient sensor;
c. Mounting an orientation sensor relative to a surgical device;
d. Sensing the orientation of the surgical device with the orientation sensor;
e. Monitoring the orientation of the surgical device relative to the sensed patient's anatomy with a monitor;
f. Providing an optimal orientation of the surgical device; and
g. Guiding the monitored surgical device to the optimal orientation with a communicator.

In one embodiment, the method first comprises securing the patient to an operating table. The patient's anatomy may be especially substantially immobilised before the surgery.

Before mounting the patient sensor and the orientation sensor, it may be advantageous to place both the patient sensor and the orientation sensor on the same plane (for example, this may involve placing the sensors side by side on a table or in a cradle). In this step, the sensors may be calibrated so that they share the same orientation readings. After the patient sensor has been mounted relative to the patient's anatomy, the patient sensor may be “zeroed”, and any change in orientation readings in zeroing the sensor may be reflected in (or transferred to) the orientation sensed by the orientation sensor. The sensors may then remain activated until the surgical device has been used or inserted. Alternatively, the surgical system also may be used without calibrating the sensors.

In another embodiment, the method comprises providing a monitor for monitoring the orientation of the surgical device relative to the sensed patient's anatomy. The monitor may be as discussed above in relation to the first and second aspects of the present invention.

In another embodiment, the patient's anatomy is the pelvis, and the step of providing an optimal orientation of the surgical device includes determining a patient's pelvic tilt angle. The pelvic tilt angle may be determined from an X-ray image of the patient's pelvis. This may be as discussed in relation to the second aspect of the present invention.

In a further embodiment, the step of guiding the monitored surgical device comprises communicating to a surgeon the orientation of the monitored surgical device relative to optimal orientation, or the difference between the optimal orientation of the surgical device and the monitored orientation of the surgical device relative to the patient's anatomy. A communicator may be used for this, and the communicator may be as described above in relation to the first and/or second aspects of the present invention. The step of determining the difference between the optimal orientation of the surgical device and the monitored orientation of the surgical device relative to the patient's anatomy may be performed by the monitor or the communicator, especially the monitor.

In a further embodiment, the patient's anatomy is the pelvis, and the surgical device is an acetabular reamer.

In another embodiment, the patient's anatomy is the pelvis, and the surgical device is an acetabular cup. In this embodiment, the method may further comprise percussively placing the acetabular cup in the acetabulum.

The method may also comprise using a support for the surgical device. For example, a support may be connected to an operating table by, for example, a bayonet connection to a fitting mounted on the operating table attachment rail. The support may be adjustable, for example using clamps, to allow positioning of the surgical device as required. If the surgical device is a reamer, for example, the support may be used to support and guide the reamer during the surgical operation. The support may also include a slide for guiding the surgical device.

The method of this aspect may be used for total hip replacement surgery. As discussed above, hip replacement surgery may include: (i) severing the femoral head and dislocating the head of the femur from the acetabulum; (ii) reaming the acetabulum to fit an acetabular cup; (iii) inserting the acetabular cup; (iv) shaping the femoral canal, and then fitting a prosthetic femoral component into the canal; and (v) fitting the prosthetic femoral component to the acetabular cup. The method of this aspect may be advantageously performed to orient the surgical device used in steps (ii) and (iii) relative to the patient's anatomy (the pelvis).

Part or all of the methods described in the fourteenth and fifteenth aspects of the present invention may be performed on a computer-readable storage medium. A monitor may comprise this medium. In one embodiment of these methods, the monitored and/or sensed measurements are recorded during the surgery. In a further embodiment of these methods, the use of the system is logged, such that the number of operations performed using the system is counted.

Features of the fourteenth and fifteenth aspects of the present invention may be as described above for the first or second aspect.

In a sixteenth aspect, the present invention provides a method of driving a prosthetic component into a patient's anatomy, the method comprising providing a prosthetic component and a placement device which comprises a driver capable of generating at least one impulse to drive the prosthetic component into a patient's anatomy; and using the device to drive the prosthetic component into the patient's anatomy. In one embodiment, the patient's anatomy is the pelvis and the prosthetic component is an acetabular cup. The placement device of the fourth aspect of the present invention or the system of the fifth aspect of the present invention may be used in this method.

In a seventeenth aspect, the present invention provides a method of guided reaming of a patient's anatomy, the method comprising sensing the orientation of a reamer relative to a patient's anatomy. The system of the third aspect of the present invention may be used in this method.

The methods of the sixteenth and seventeenth aspects of the present invention may be performed using the steps described above for the fourteenth and fifteenth aspects of the present invention.

In an eighteenth aspect, the present invention provides a patient sensor when used in the method of the fourteenth or fifteenth aspects of the present invention.

In a nineteenth aspect, the present invention provides an orientation sensor when used in the method of the fourteenth or fifteenth aspects of the present invention.

In a twentieth aspect, the present invention provides a monitor when used in the method of the fourteenth or fifteenth aspects of the present invention.

In a twenty-first aspect, the present invention provides a communicator when used in the method of the fourteenth or fifteenth aspects of the present invention.

In a twenty-second aspect, the present invention provides computer software for use in the system of the first or second aspects of the present invention, or when used in the method of the fourteenth or fifteenth aspects of the present invention.

A person skilled in the art will appreciate that many embodiments and variations can be made without departing from the ambit of the present invention.

In the present specification and claims, the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

Any of the features described herein can be combined in any combination with any one or more of the other features described herein within the scope of the invention.

### BRIEF DESCRIPTION OF DRAWINGS

Preferred features, embodiments and variations of the invention may be discerned from the following Detailed Description which provides sufficient information for those skilled in the art to perform the invention. The following Detailed Description is not to be regarded as limiting the scope of the preceding Summary of Invention in any way. The Detailed Description will make reference to a number of drawings as follows:

FIG. 1 is a schematic diagram of a surgical system;

FIG. 2 is a schematic diagram of a surgical system, showing a patient's anatomy and a surgical device;

FIG. 3 is a flowchart of a method of orienting a surgical device relative to a patient's anatomy;

FIG. 4 is an elevation view of a sensing portion of an orientation sensor according to one example of the present invention;

FIG. 5 is a plan view of the sensing portion of the orientation sensor shown in FIG. 4;

FIG. 6 is an elevation view of a sensing portion of a patient sensor according to another example of the present invention;

FIG. 7 is a plan view of the sensing portion of the patient sensor shown in FIG. 6;

FIG. 8 is a side view of an orientation sensor mounting mounted to a placement device for placing an acetabular cup according to a further example of the present invention;

FIG. 9 is a top view of the orientation sensor mounting shown in FIG. 8;

FIG. 10 is a rear view of the orientation sensor mounting shown in FIG. 8;

FIG. 11 is a plan view of part of the shock absorber of the orientation sensor mounting shown in FIG. 8;

FIG. 12 is a side view of an orientation sensor mounting mounted to a surgical device (an acetabular reamer) according to another example of the present invention;

FIG. 13 is a top view of the orientation sensor mounting shown in FIG. 12;

FIG. 14 is a rear view of the orientation sensor mounting shown in FIG. 12;

FIG. 15 is a cross-sectional view through section A-A, as shown in FIG. 12;

FIG. 16 is a top view of a patient sensor base, according to a further example of the present invention;

FIG. 17 is a cross-sectional view through section A-A, as shown in FIG. 16;

FIG. 18 is a side view of the patient sensor base shown in FIG. 16;

FIG. 19 is a side view of the patient sensor base shown in FIG. 16, also showing a patient sensor fastener;

FIG. 20 is an exemplary output from a monitor/communicator, showing how deviations in the patient position on the operating table and anatomical deviations in the patient may be inputted to adjust the optimal orientation angle;

FIG. 21 is an exemplary output from a monitor/communicator, showing the pitch, roll and yaw of the orientation sensor relative to the patient sensor;

FIG. 22 is an exemplary output from a monitor/communicator, showing the pitch, roll and yaw of the orientation sensor relative to the patient sensor;

FIG. 23 is a perspective view of an orientation sensor mounted to a surgical device (an acetabular reamer) according to another example of the present invention;

FIG. 24 is a perspective view of an orientation sensor mounted to a placement device for placing an acetabular cup according to another example of the present invention;

FIG. 25 is a perspective view of an orientation sensor mounted to a surgical device (an acetabular reamer) according to another example of the present invention;

FIG. 26 is a top view of a clamp nut for securing an orientation sensor clamp to a spacer according to another example of the present invention;

FIG. 27 is a side view of the clamp nut of FIG. 27;

FIG. 28 is a side view of an orientation sensor spacer according to another example of the present invention;

FIG. 29 is an end view of the orientation sensor spacer of FIG. 28;

FIG. 30 is a top view of an orientation sensor docking port for securing to an orientation sensor spacer according to a further example of the present invention;

FIG. 31 is a side view of the orientation sensor docking port of FIG. 30;

FIG. 32 is a side view of a clamp bolt for securing an orientation sensor docking port to an orientation sensor spacer according to a further example of the present invention;

FIG. 33 is a top view of the clamp bolt of FIG. 32;

FIG. 34 is a side view of an orientation sensor clamp mounted to a placement device for placing an acetabular cup according to a further example of the present invention;

FIG. 35 is an end view of the orientation sensor clamp of FIG. 34;

FIG. 36 is a plan view of part of the shock absorber for the orientation sensor clamp of FIG. 34;

FIG. 37 is a side view of a fastener for fastening the orientation sensor clamp of FIG. 34 to a placement device for placing an acetabular cup;

FIG. 38 is a top view of the fastener of FIG. 37;

FIG. 39 is a side view of an orientation sensor clamp mounted to a surgical device (an acetabular reamer) according to another example of the present invention;

FIG. 40 is a bottom view of the orientation sensor clamp of FIG. 39;

FIG. 41 is a plan view of a bolt for fastening the orientation sensor clamp of FIG. 39 to a surgical device;

FIG. 42 is a side view of a nut for fastening the orientation sensor clamp of FIG. 39 to a surgical device;

FIG. 43 is a side view of the nut of FIG. 42;

FIG. 44 is a side view of an orientation sensor sensing portion housing according to another example of the present invention;

FIG. 45 is a plan view of a docking port for an orientation sensor according to another example of the present invention;

FIG. 46 is an end view of the body of the housing of FIG. 44;

FIG. 47 is an end view of the body of the housing of FIG. 44;

FIG. 48 is a plan view of the body of the housing of FIG. 44;

FIG. 49 is a plan view of the lid of the housing of FIG. 44;

FIG. 50 is a top view of a patient sensor base, according to a further example of the present invention;

FIG. 51 is a cross-sectional view through section A-A, as shown in FIG. 50; and

FIG. 52 is a cross-sectional view through section B-B, as shown in FIG. 50.

### DETAILED DESCRIPTION OF EMBODIMENTS

In the figures, like reference numerals refer to like features.

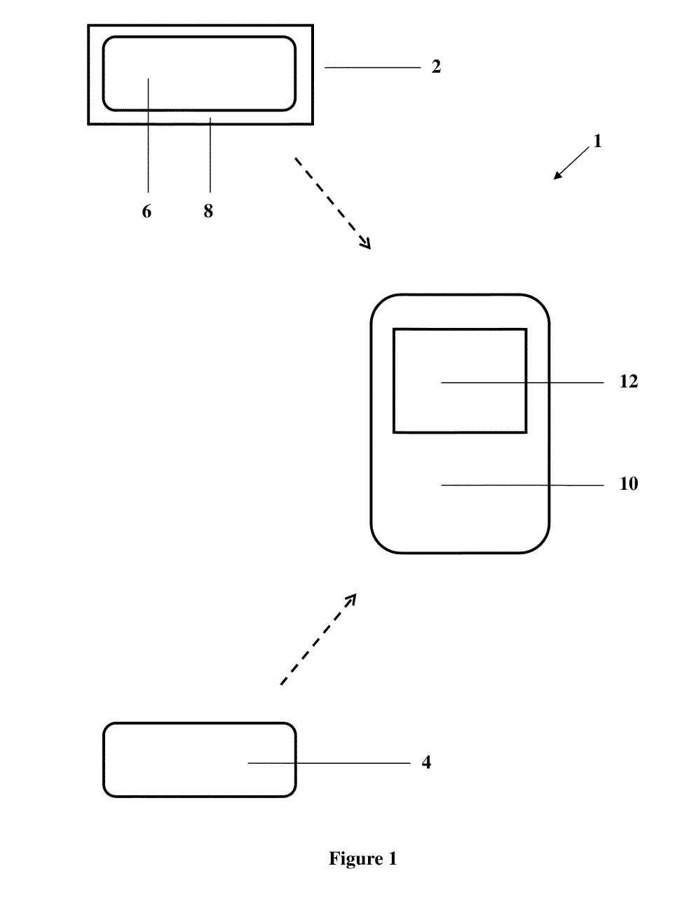

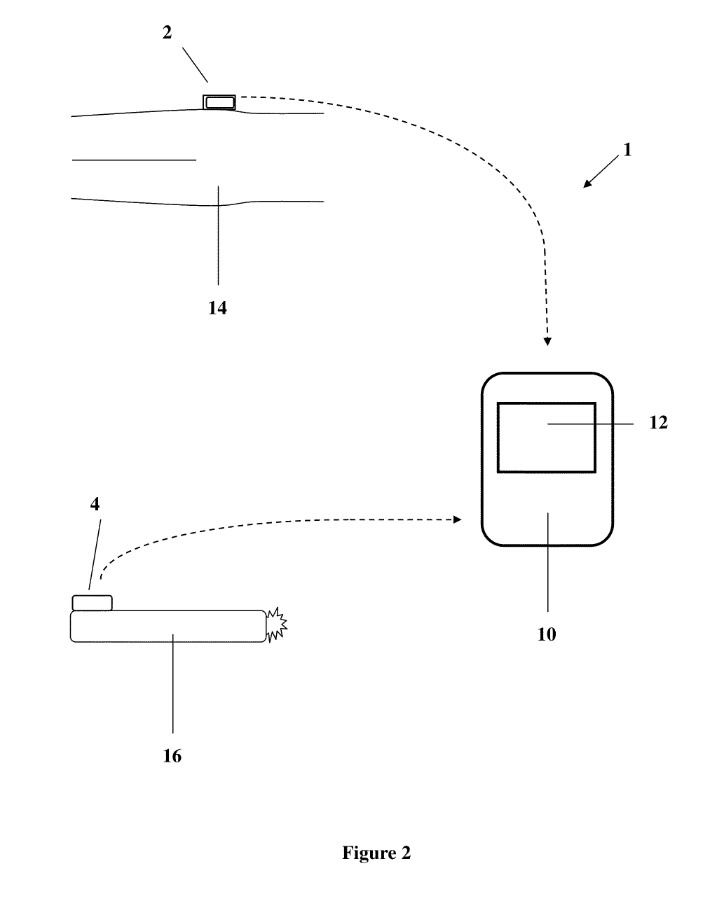

As illustrated in FIGS. 1 and 2, in an embodiment of the present invention there is provided a surgical system 1 for monitoring the orientation of a surgical device 16 relative to a patient's anatomy 14. The system 1 comprises: a patient sensor 2 for sensing the orientation of the patient's anatomy 14; an orientation sensor 4 for sensing the orientation of a surgical device 16; and a monitor 10 for monitoring the orientation of the surgical device relative to the sensed patient's anatomy. In the example of FIG. 2, the patient's anatomy 14 is the pelvis, and the surgical device 16 is an acetabular reamer.

The system 1 illustrated in FIGS. 1 and 2 is for surgery on the hip, especially in hip reconstruction, and most especially in total hip replacement. In this system, the orientation of the patient's pelvis 14 is sensed by the patient sensor 2. In FIG. 2, the patient sensor 2 is shown mounted to the patient's iliac crest.

The patient sensor 2 may comprise a sensing portion 6 and a base 8, in which the sensing portion 6 is mountable to the base 8. The base 8 may be mountable to the patient's pelvis 14 by a fastener in the form of adhesive, strapping, pedicle screws, or Velcro™ dot fasteners.

The sensing portion 6 may sense one or more of pitch, roll and yaw, especially all of pitch, roll and yaw. The sensing portion 6 may comprise at least one sensor for sensing the pitch, roll and/or yaw of the patient's pelvis.

The sensing portion 6 may also comprise an integrated or external power supply, in the form of a battery. The battery may be replaceable or rechargeable.

The sensing portion 6 and/or the base 8 may be disposable or sterilisable.

The system 1 also comprises an orientation sensor 4 which may be mountable to a surgical device 16. FIG. 2 illustrates the orientation sensor 4 mounted to a surgical device 16 which is a surgical implement, especially an acetabular reamer. In another embodiment, the surgical device may be a prosthetic component, especially a prosthetic component for hip replacement surgery, most especially an acetabular cup, in which case the orientation sensor 4 may be mounted relative to the surgical device 16.

In one embodiment, the orientation sensor 4 is mountable to a combination surgical power device 16 for reaming and subsequent placement of a prosthetic component, especially for variable speed reaming and variable impulse placement of a prosthetic component. The orientation sensor may be mounted to a docking port on the power device 16, the docking port set at an appropriate angle and adjustable inclination to the axial line. Preferably, the docking port may be lockable by deforming a portion of the port. For example, the docking port may comprise a flexible plastic hinge between the docking port door and the remainder of the port. The docking port door may be closed by bending the flexible plastic.

The device 16 in this embodiment may have a detachable head. This allows an acetabular reamer, for example, to be fitted to the device 16 when the patient's acetabulum is to be reamed, and after reaming is completed the reamer output drive shaft may be replaced with a driver for driving an acetabular cup into the acetabulum. This may be achieved by a snap connection compatible with existing in use surgical devices. The driver may be capable of generating at least one impulse, especially multiple impulses, to drive the acetabular cup into the acetabulum. The timing of the impulses may be adjustable, and the driver may be capable of detecting when the acetabular cup cannot be driven further into the acetabulum. For example, this may be achieved by detecting the movement of the acetabular cup relative to the device following an impulse. The device in this embodiment may generate impulses by, for example, piezoelectric action, or by use of the action of compressing a spring with a power operated cam and quick release mechanism. The impulse also may be generated pneumatically, hydraulically or by a motor, and preferably the impulse may be generated pneumatically.

The acetabular cup may be mounted to the device by a quick action coupling (which may be spring loaded) which engages with a range of suitable length shafts each with a different thread to suit the various acetabular cups used by the surgeon.

Alternatively, a separate reamer and a placement device comprising a driver as discussed above may be used. The orientation sensor may be mountable relative to the reamer and the placement device.

The orientation sensor 4 may be placed in any suitable position relative to the surgical device 16. In one example, the orientation sensor 4 is mountable to a docking port on or relative to the surgical device 16. The docking port is especially lockable by deforming a portion of the port.

The orientation sensor 4 may sense one or more of pitch, roll and yaw, especially all of pitch roll and yaw. The orientation sensor may comprise at least one sensor for sensing the pitch, roll and/or yaw of the surgical device 16.

The orientation sensor 4 may also comprise an integrated or external power supply, in the form of a battery. The battery may be replaceable or rechargeable.

The orientation sensor may be disposable or sterilisable. If the patient sensor and/or the orientation sensor are sterilisable, then the patient sensor and/or the orientation sensor may further comprise an insulator for insulating the electronic components of the sensor from chemical, thermal or pressure effects. The insulator may provide insulation from the influence of standard autoclave sterilising or alternatively a “gas” approved sterilisation process (which may be low temperature). In one embodiment, the insulator is a casing.

The surgical system 1 also comprises a monitor 10 for monitoring the orientation of the surgical device relative to the sensed patient's anatomy. The monitor 10 may be in one or two way communication with the patient sensor 2 and the orientation sensor 4, to receive the sensed orientation of the patient's anatomy and the sensed orientation of the surgical device. In the system 1 illustrated in FIGS. 1 and 2 the patient sensor 2 and the orientation sensor 4 are in wireless communication with the monitor 10.

The surgical system 1 may also comprise a communicator 12. In FIGS. 1 and 2 the communicator 12 is a screen of a computer and the computer also comprises a monitor 10. The computer may be remote to the patient sensor 2 and the orientation sensor 4. The communicator 12 may be for visually communicating the sensed orientation of the patient's anatomy, the sensed orientation of the surgical device and the monitored orientation of the surgical device relative to the patient's anatomy. The communicator 12 may include a graphical display.

In the embodiment illustrated in FIGS. 1 and 2, the system 1 is also for orienting a surgical device 16 relative to a patient's anatomy 14. In addition to the features discussed above, the communicator 12 may be for guiding the monitored surgical device to an optimal surgical device orientation relative to the patient's anatomy.

In some embodiments, an optimal orientation of the surgical device relative to the patient's anatomy is provided to the monitor 10 or determined by the monitor 10. The optimal orientation may involve consideration of the patient's pelvic tilt angle. The monitor 10 or communicator 12 may also determine the difference between the optimal orientation of the surgical device and the monitored orientation of the surgical device relative to the sensed patient's anatomy. The communicator 12 may guide the monitored surgical device by communicating this difference to the surgeon.

In the embodiment illustrated in FIGS. 1 and 2, the communicator guides the monitored surgical device by communicating to the surgeon the sensed orientation of the patient's anatomy, the sensed orientation of the surgical device, the monitored orientation of the surgical device relative to the patient's anatomy, the optimal orientation of the surgical device, and the difference between the optimal orientation of the surgical device and the monitored orientation of the surgical device relative to the patient's anatomy. The communicator 12 illustrated in FIGS. 1 and 2 communicates visually with the surgeon via a visual display. However, the communicator may also communicate audibly with the surgeon.

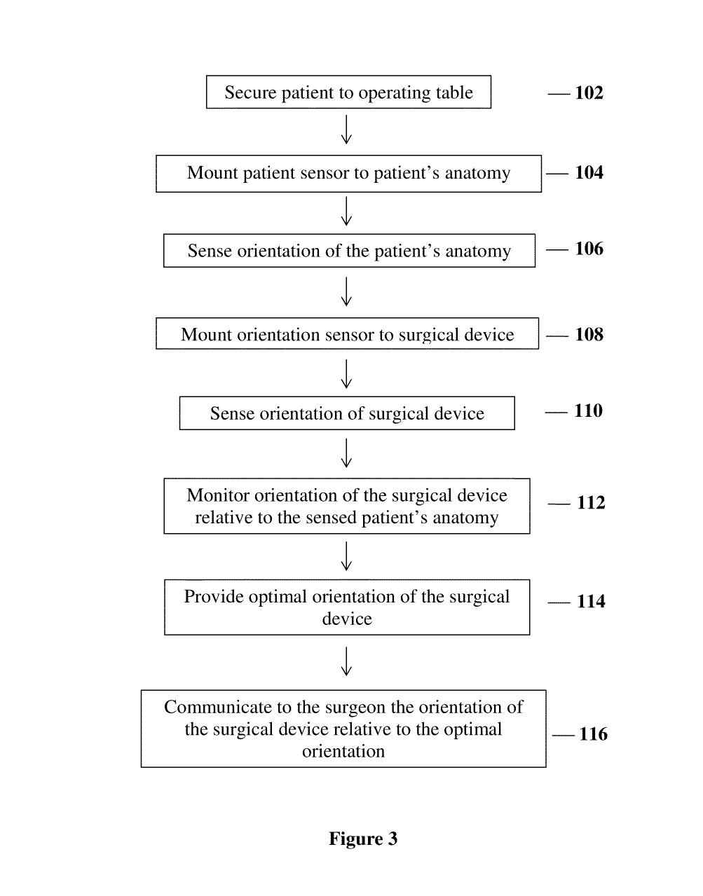

In another embodiment, the present invention relates to a method of monitoring the orientation of a surgical device relative to a patient's anatomy, the method comprising: sensing the orientation of the patient's anatomy 106, and monitoring the orientation of the surgical device relative to the sensed patient's anatomy 112. This is illustrated in the flowchart in FIG. 3.

In a further embodiment, the invention relates to a method of orienting a surgical device relative to a patient's anatomy. This method comprises: sensing the orientation of the patient's anatomy 106, monitoring the orientation of the surgical device relative to the sensed patient's anatomy 112, providing an optimal orientation of the surgical device 114 and guiding the monitored surgical device to the optimal orientation. This method is illustrated in further detail in FIG. 3. The flowchart illustrated in FIG. 3 is discussed below in relation to an embodiment of the present invention, which relates to a total hip replacement operation.

First, a patient is secured to the operating table 102. In a hip replacement operation, the patient may be positioned with the hip to be replaced vertically above the opposite hip. Care should be taken to move the patient's pelvis anteriorly and/or posteriorly to determine a neutral pelvis position for the patient. Once the patient's pelvis is in a neutral position, the patient may be secured to the operating table so that the patient's pelvis remains in that position. This may be achieved using adjustable anterior and posterior clamps against the pelvic zone, or the patient may be secured to the operating table using, for example, adhesive tape. If anterior and posterior clamps are used, the posterior clamp should be applied to the sacrum to minimise movement in the lumbar spine. This may substantially immobilise the patient's anatomy. Positioning the patient in this way permits a lateral approach to the hip replacement operation.

However, it would be appreciated that the systems, methods and devices of the present invention may be used in other approaches to hip replacement surgery. For example, the system may be used in a posterior approach or an anterior approach. In an anterior approach for example, the patient may be positioned in a supine position, on their back (in this position, the patient sensor may be placed on the anterior superior iliac spine and/or the pubic symphysis). The patient may be secured to the operating table using, for example, adhesive tape.

The systems, methods and devices of the present invention may also be used in minimally invasive surgical procedures.

Secondly, the patient sensor may be placed side by side with the orientation sensor on a table. The sensors may be activated and then calibrated by synchronising their readings.

Thirdly, the patient sensor is mounted to the patient's anatomy 104. The patient sensor 2 may be mounted to the patient's pelvis 14, especially to the patient's iliac crest or sacrum. The patient sensor 2 may be mounted to the patient's pelvis 14 by adhesive, strapping, or pedicle screws.

The patient sensor 2 may then be “zeroed”, setting the pitch, roll and yaw to 0. This allows the orientation of the patient's anatomy to be sensed 106. Any difference in the orientation sensed by the patient sensor before and after zeroing is transferred to the readings by the orientation sensor.

After the surgeon has severed and dislocated the femoral head, the acetabulum is partly reamed to establish the centre of the replacement acetabular cup (the centre of the acetabular cup in the acetabulum may also be determined by X-ray prior to surgery). The reamer 16 is then held in the estimated reaming orientation with the cutter in the established acetabular cup position. The orientation sensor is mounted to the reamer (the surgical device) 108, and the orientation sensor senses the orientation of the surgical device 110. It is preferable to mount the orientation sensor to the reamer once the reamer is in position to minimise disturbance in sensor readings. Pitch, roll and yaw are sensed by the orientation sensor.

The orientation of the surgical device (the reamer) relative to the patient's anatomy is then monitored 112. In this way, the system 1 compensates for any patient movement during the surgical procedure. This step may be performed by monitor 10.

The optimal orientation of the surgical device (the reamer) is then provided 114, especially to the monitor 10. The optimal orientation of the reamer may be especially determined by the monitor 10. The optimal orientation of the reamer may be determined by considering the pelvic tilt angle, and this may be determined by X-ray. Consideration of the pelvic tilt angle may involve manually adjusting the optimal orientation or the deviation may be recorded as offsets.

Next, the orientation of the surgical device relative to the optimal orientation is communicated to the surgeon 116. This may be performed by the communicator 12.

In a first example, the communicator 12 may visually communicate with the surgeon. In this example, the communicator 12 may be positioned within the surgeon's line of sight during the operation, and may show the monitored orientation of the reamer relative to the patient's anatomy, and the optimal orientation of the reamer. This may allow the surgeon to visually observe the difference between the monitored orientation and the optimal orientation, and make corrections to bring the reamer 16 into the optimal orientation.

In a second example, the communicator 12 may audibly communicate with the surgeon. In this example, the surgeon is able to hear the communicator 12, which guides the surgeon by using sounds of different pitch, tone and/or duration. For example, when the reamer 16 is not in the optimal orientation the communicator 12 may make a beeping sound, and when in the correct orientation the communicator 12 may make a pinging sound.

In a third example, the communicator 12 may communicate with the surgeon both visually and audibly, as described in relation to the first and second examples above.

Consequently, the communicator 12 guides the surgeon in orienting the reamer to the optimal position. Reaming is completed as guided by the communicator 12.

When performing reaming, the surgeon may find it convenient to use a support. The support, for example, may be fitted to the support guide rail of an operating table to enable it to be correctly aligned. The support may be fitted by a bayonet connection, for example. The support may be adjustable, for example using clamps, to allow positioning of the surgical device as required. The reamer engages with a support slide on the support to complete the reaming operation. The support may also be used when fitting the acetabular cup.

After reaming is completed, the surgical system may be used for monitoring and orienting an acetabular cup relative to the patient's pelvis. If using a combination power device, the device is switched to impulse mode and the reaming cutter is replaced with an acetabular cup holder and drive bar. The acetabular cup is oriented in the acetabulum by steps 112, 114 and 116. Alternatively, the orientation sensor may be mounted to a separate impulse device for inserting the acetabular cup, and the cup may be oriented either by the steps illustrated in FIG. 3 and discussed above, or by using the support slide with the same setting used for reaming.

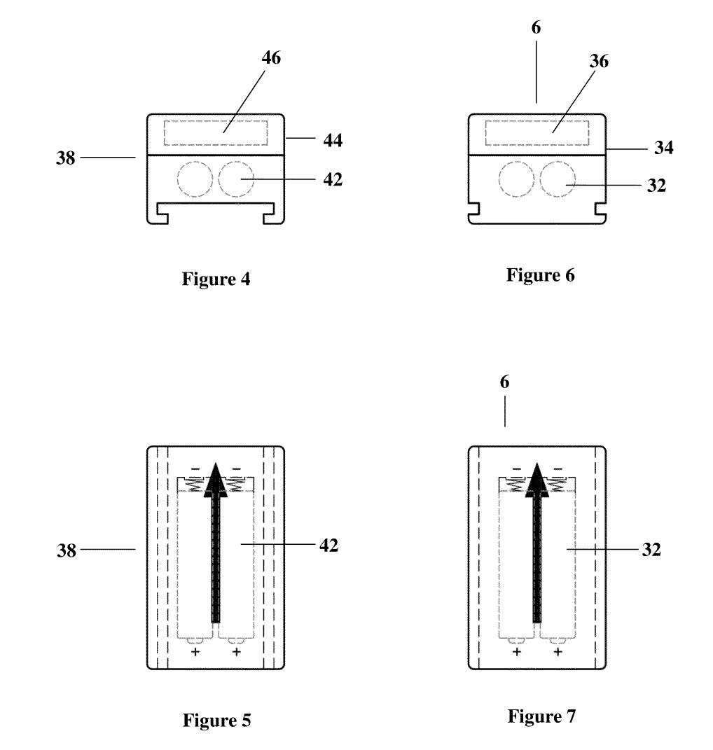

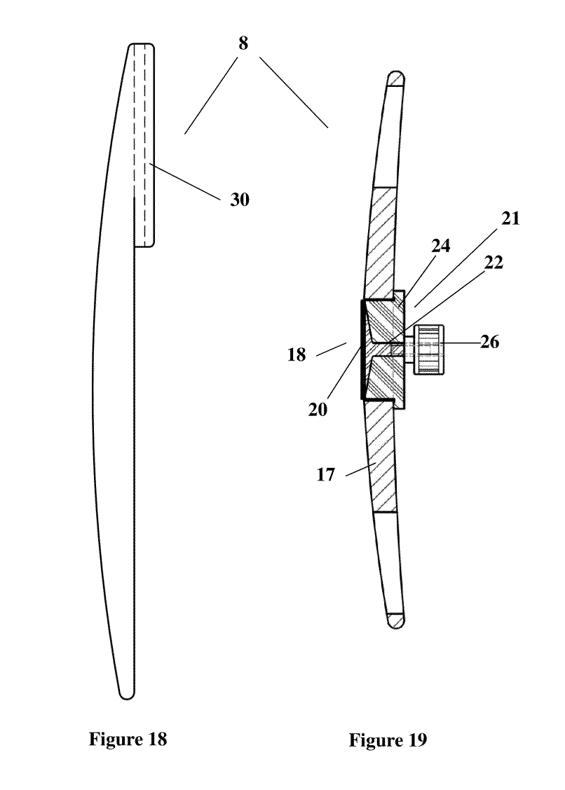

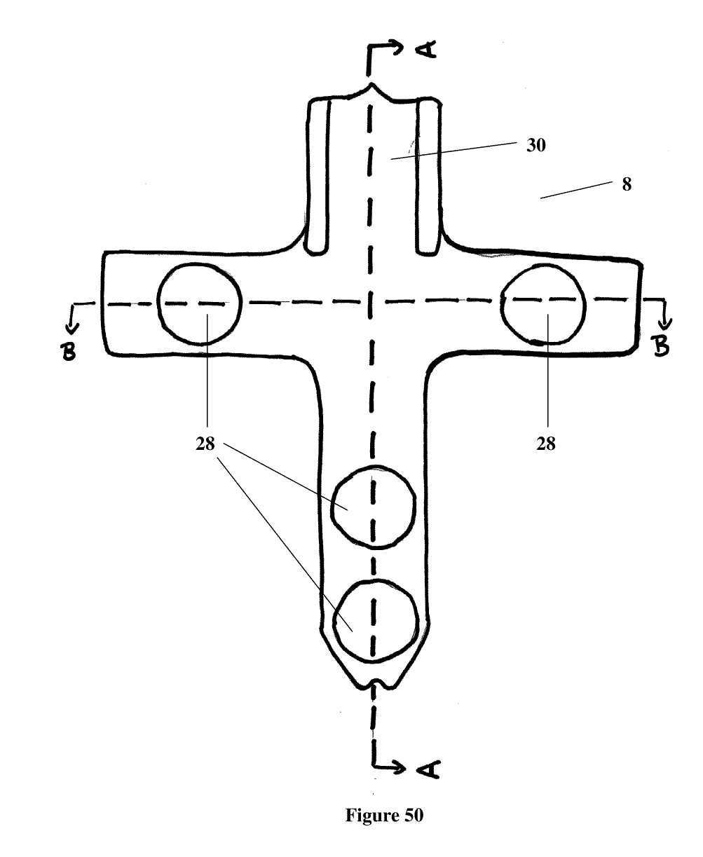

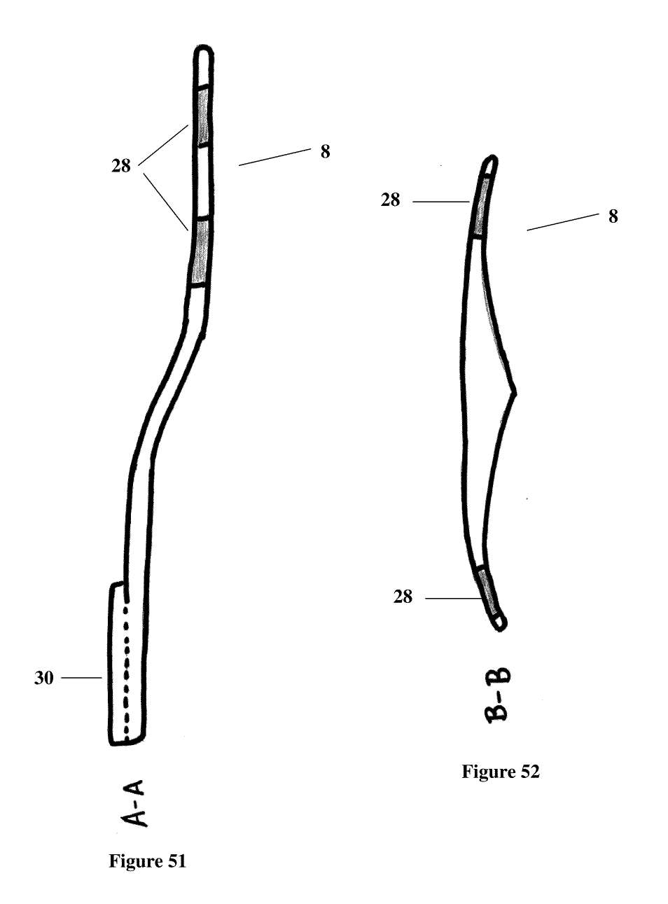

Further examples of the present invention are illustrated in FIGS. 4-19 and 23-52. These figures provide surgical systems for monitoring the orientation of a surgical device relative to a patient's anatomy, especially for surgery on the hip, especially total hip replacement.

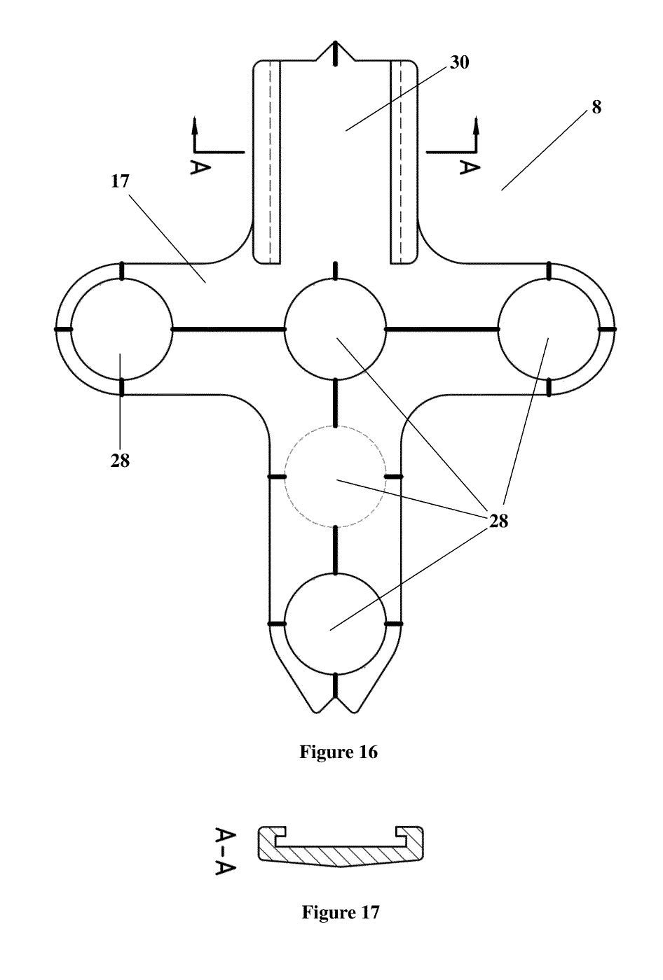

The patient sensor 2 comprises a sensing portion 6 (see FIGS. 6 and 7) and a base 8 (see FIGS. 16-19). The base 8 is designed to be mountable to the sacrum zone of the patient. The base may comprise a body 17 and at least one coupler 18, wherein the body 17 is mountable to the at least one coupler 18, and the at least one coupler 18 may be mounted relative to the patient's anatomy (see FIG. 19). The coupler 18 illustrated in this figure comprises a fastener 20 (in the form of an adhesive layer) for mounting the coupler 18 relative to the patient's sacrum. While any suitable type of adhesive may be used, the adhesive may be especially a similar type to those used on the electrodes when performing electrocardiography (ECG) (and may, for example, have a diameter of approximately 30 mm) The coupler 18 also comprises a clamp 21, which comprises a threaded rod 22 (especially an approximately 4 mm stainless steel thread), a securing collar 24 and a nut 26 (which is especially knurled).

The base 8 may define any number of apertures 28, especially two, three, four, five or six apertures 28; more especially four or five apertures 28. The example illustrates in FIG. 16 has four or five apertures 28 (the aperture 28 marked with a dotted line may be present or absent). Each aperture may accommodate a coupler 18, as discussed above. The couplers 18 may be positioned after the alignment of the base 8 is completed.

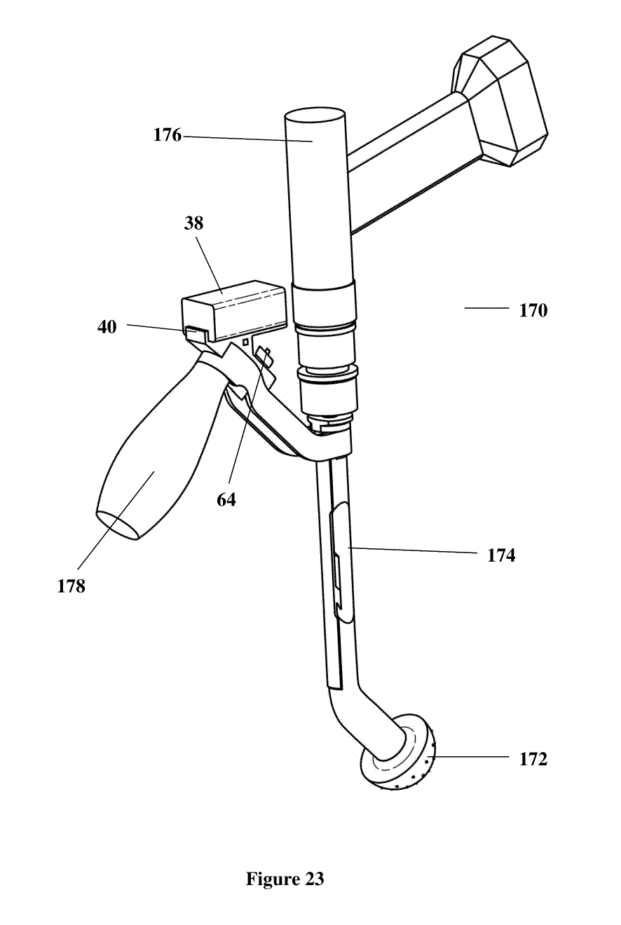

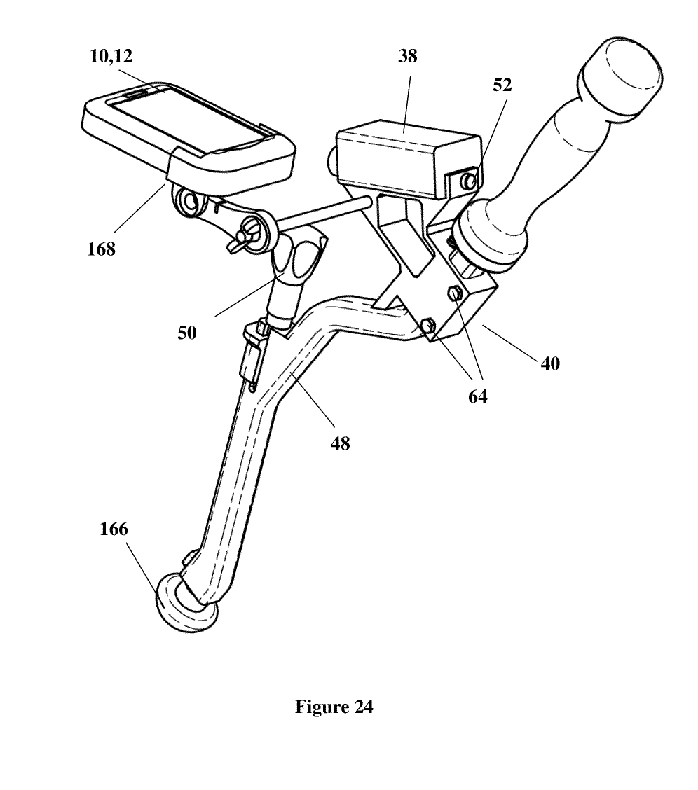

At one end of the base 8 is a docking port 30, to which the patient sensor sensing portion 6 may be mounted (see FIGS. 6, 7 and 16). As shown in FIGS. 6, 16 and 17, the sensing portion 6 possesses a groove which complements the shape of the docking port 30, so that the sensing portion 6 may be slidably engaged in the docking port 30. However, the sensing portion 6 may also be screwed into the docking port 30 in the manner illustrated for the orientation sensor sensing portion 38 in FIGS. 24 and 25. The base 8 and sensing portion 6, when coupled together in the sacrum zone of a patient, are designed so that the arrow on the patient sensor sensing portion 6 points towards the patient's head. Once the base 8 and sensing portion 6 (together the patient sensor 2) are positioned correctly, the surgeon may further secure the patient sensor 2 to the patient using surgical grade Elastoplast® tape. It should be noted that the patient sensor 2 is especially independent of the pelvic brace used to secure the patient to the operating table.

A further example of a patient sensor base 8 is illustrated in FIG. 50-52. These Figures illustrate a base 8 with a docking port 30 (for mounting the patient sensor sensing portion 6 illustrated in FIGS. 6 and 7) and four apertures 28, and each of the apertures may accommodate a coupler 18, as discussed above. This base 8 is contoured to the shape of a patient's sacrum.

The sensing portion 6 includes a power supply 32 (in the form of one or two batteries), a housing 34, and a sensor 36. The battery (or batteries) is especially sterilisable, and most especially is a lithium ion battery (e.g. 3.6V). The battery may be rechargeable or non-rechargeable. The sensor 36 is able to measure orientation in three axes (pitch, roll and yaw), and contains a wireless data transmission (such as Blue Tooth™ or Wi-Fi connection). The sensor 36 may include pitch and roll sensors with 2.4 GHz wireless transmission, and a yaw sensor with an ultra-sensitive magnetometer (in one example, the yaw sensor may be a mechanical yaw detector which converts the measured yaw to a second pitch sensor which is translated to a yaw tool indication). The sensor 36 may measure approximately 40×28×70 mm, be non-magnetisable and be able to be quickly and securely positioned in the sensing portion 6. The sensing portion 6 may also include an indicator, such as a light, for indicating the power remaining in the power supply 32.

The patient sensor 2 may be made of any suitable material, and the base is especially made from a plastic such as polytetrafluoroethylene (PTFE). The base is especially made of an X-ray translucent material. Of course, a range of shapes may be used to suit individual patients, and the area to which the patient sensor 2 is to be mounted. All components of the patient sensor 2, with the possible exceptions of the sensor 36 and the power supply 32, may be sterilisable. The sensor 36 and the power supply 32 may not require sterilisation if they are fully encased in a sterile housing 34.

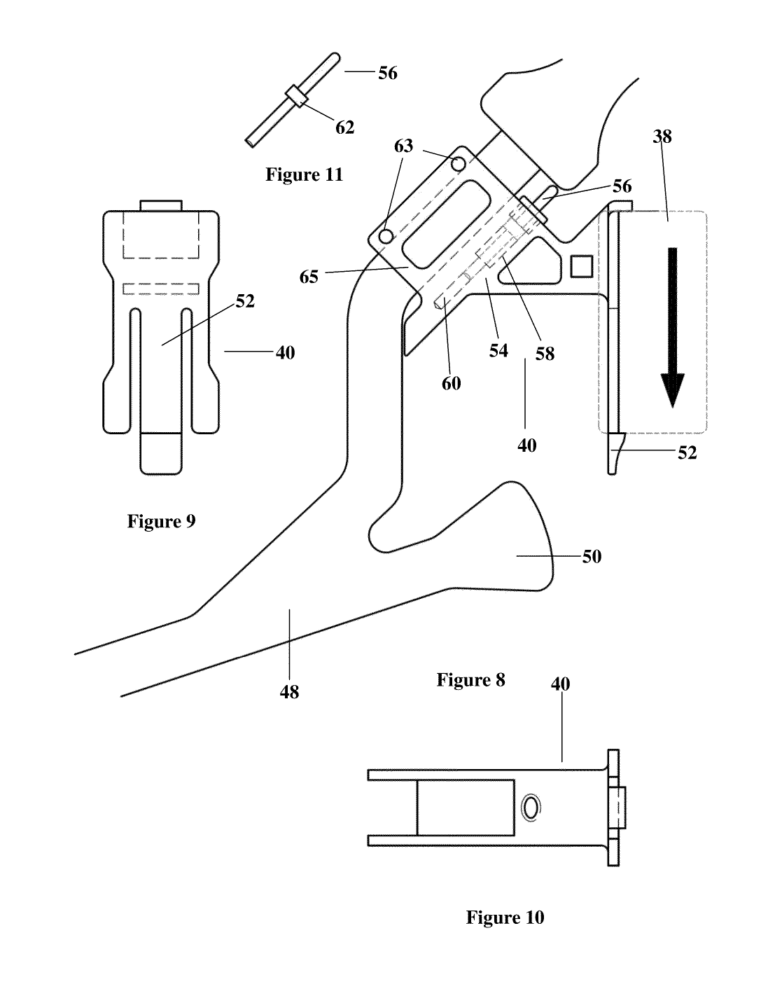

The system 1 also comprises an orientation sensor 4, which comprises a sensing portion 38 (see FIGS. 4 and 5) and a mounting 40 (illustrated in FIGS. 8-10 and 12-14). The sensing portion 38 includes power supply 42 (in the form of battery), a housing 44, and a sensor 46. The battery (or batteries) is especially sterilisable, most especially is a lithium ion battery (e.g. 3.6V). The battery may be rechargeable or non-rechargeable. The sensor 46 is able to measure orientation in three axes (pitch, roll and yaw), and contains a wireless data transmission (such as Blue Tooth™ or Wi-Fi connection). The sensor 46 may include pitch and roll sensors with 2.4 GHz wireless transmission, and a yaw sensor with an ultra-sensitive magnetometer. The sensor 46 may measure approximately 40×28×70 mm, be non-magnetisable and be able to be quickly and securely positioned in the sensing portion 38. The sensing portion 38 may also include an indicator, such as a light, for indicating the power remaining in the power supply 42. The sensors 46 and 36 may be identical.

As shown in FIG. 5, the sensing portion 38 especially has an arrow pointing in the sensor pairing direction which will, when attached to the mounting 40, be the direction towards the patient's head when in use.

Two different mountings 40 are illustrated in FIGS. 8-10 and 12-15. It would be appreciated that any suitable design for the mounting 40 may be used, and the design will vary depending on the component to which the mounting 40 is to be fixed.

The mounting illustrated in FIGS. 8-10 is to be attached to a placement device 48 for placing an acetabular cup. This placement device 48 is a curved acetabular cup inserter (the knob 50 is for releasably coupling the acetabular cup). An exemplary acetabular cup inserter is manufactured by DePuy (Catalogue Number 920010029). FIG. 24 illustrates the DePuy acetabular cup inserter 48, together with an acetabular cup 166, and a knob 50 for releasably coupling the acetabular cup.

The mounting 40 includes a docking port 52 which is complementary in shape to sensing portion 38. Bending the tongue of the docking port 52 allows the sensing portion 38 to be uncoupled. The mounting 40 is arranged for quick acting positive position locking of the sensing portion 38.

The mounting 40 also includes a shock absorber 54 (see FIGS. 8 and 11). The shock absorber 54 may be biased against the handle of the placement device 48, and may be assembled with an impact shaft (spool) 56 (preferably titanium), a coil spring 58 (preferably stainless steel), and a pneumatic compression chamber 60. A collar 62 on the titanium spool 56 may abut the spring 58, and the end of the titanium spool 56 may extend partway into the compression chamber 60. However, the pneumatic compression chamber 60 is optional. Other types of shock absorbers may be used, such as a two-direction shock absorber.

The mounting 40 may include a clamp 65 for clamping the mounting to the placement device. The clamp includes a fastener 63 such as bolts and lock nuts. The mounting 40 may be made from a sterilisable material, and the body of the mounting may be especially made of stainless steel (such as grade 316). The mounting 40 is especially made of a non-magnetisable material.

A different mounting 40 for an acetabular cup inserter 48 is illustrated in FIG. 24. In this figure the entire acetabular cup inserter 48 is illustrated, along with acetabular cup 166. As discussed above, knob 50 is for releasably coupling the acetabular cup 166. The mounting 40 illustrated in this figure is essentially the same as the mounting illustrated in FIGS. 8-10, except that the sensing portion 38 is screwed into the docking port 52, and that the mounting includes a bracket 168 for holding a monitor 10 and communicator 12. In FIG. 24, the monitor 10 and communicator 12 are co-located in a tablet (or an iPad®) or a smartphone (such as an iPhone®) or the like. In use, the tablet or smartphone may be encased within a sterilisable housing which is held in, or forms part of the bracket 168.

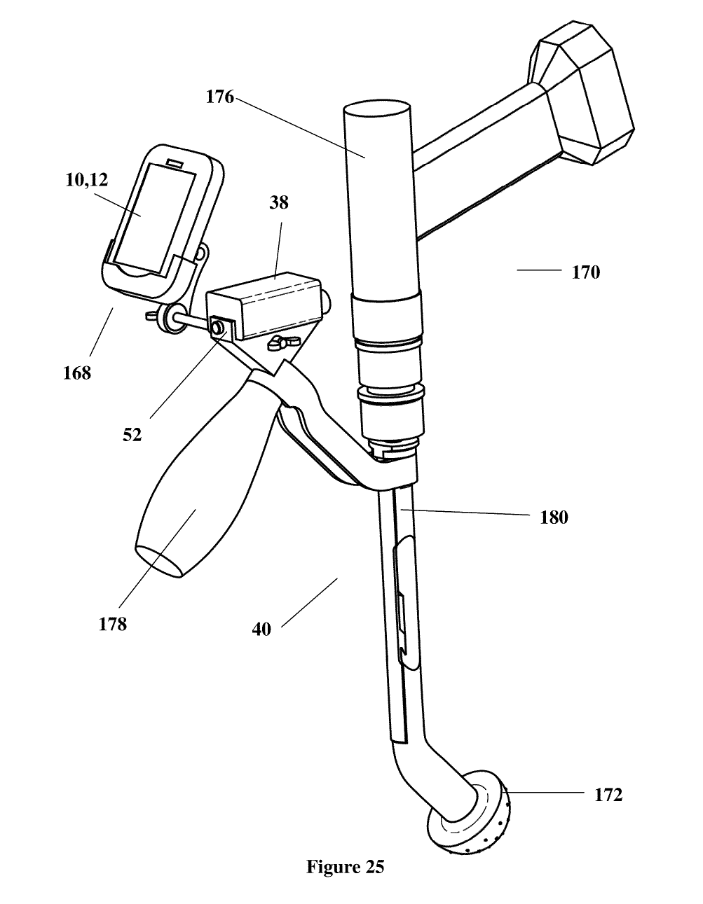

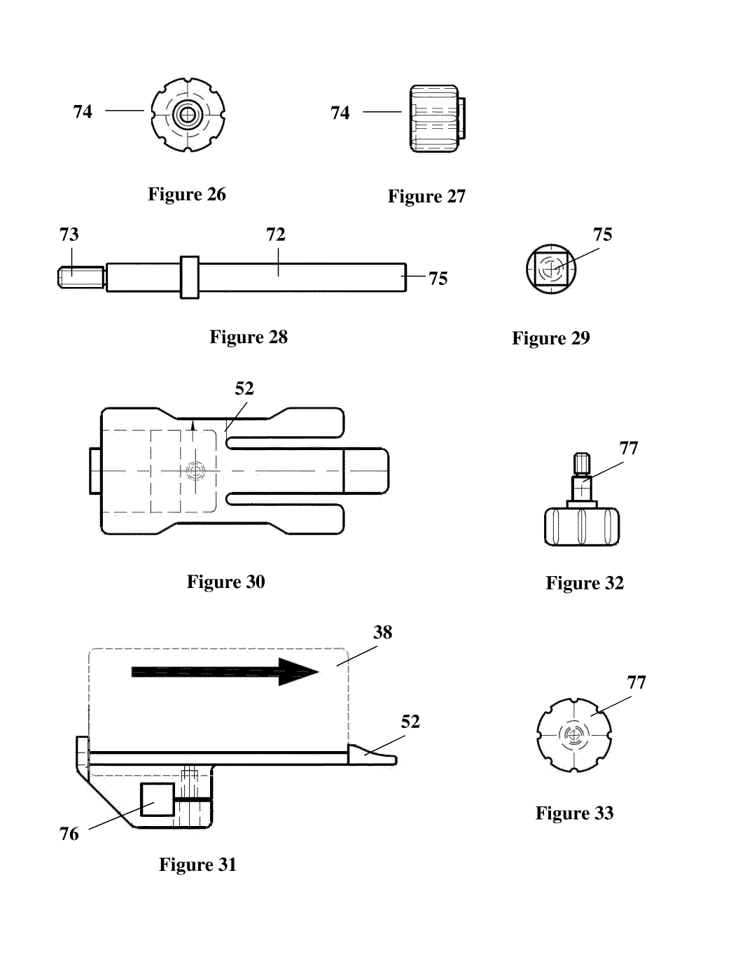

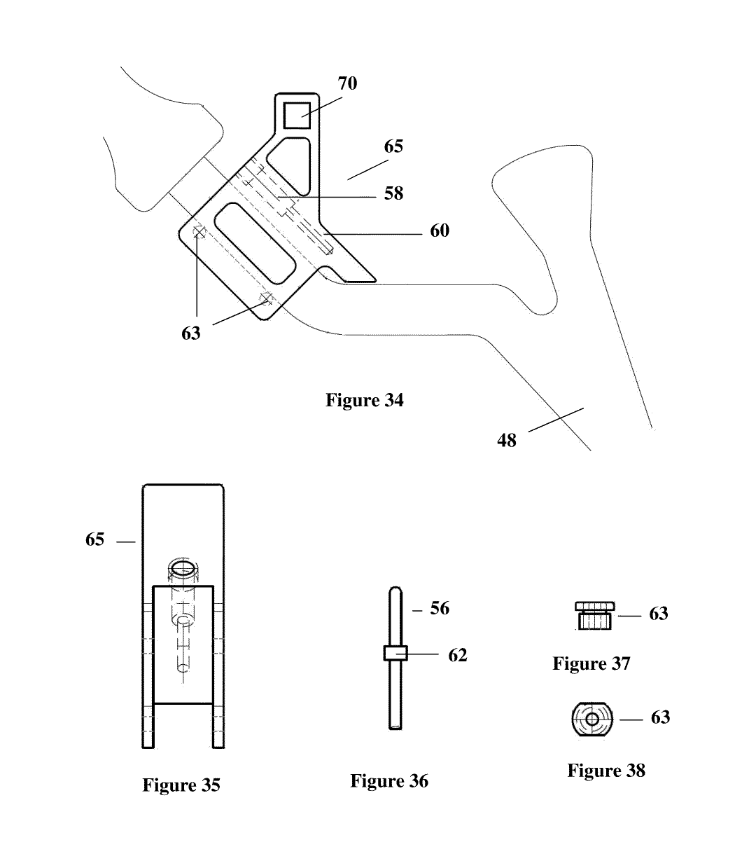

A further mounting 40 for an acetabular cup inserter 48 is illustrated in FIGS. 26-38. This mounting includes a clamp 65 (see FIG. 34), a spacer attachment region 70, and a shock absorber 54 (assembled with an impact shaft (spool) 56 with a collar 62 (see FIG. 36), a coil spring 58, and a compression chamber 60). The clamp 65 includes two fasteners 63 (see FIGS. 34, 37 and 38). A first end 73 of spacer 72 (see FIGS. 28 and 29) may be inserted into spacer attachment region 70, and secured in place via clamp nut 74 (see FIGS. 26 and 27). A second end 75 of spacer 72 may be inserted into docking port attachment region 76 (see FIG. 31) and secured in place with clamp bolt 77 (see FIGS. 32 and 33). Orientation sensor sensing portion 38 (as shown in FIGS. 4 and 5) may be mounted to docking port 52 (see FIGS. 30 and 31). Advantageously, in this arrangement a surgeon's line of sight down the central axis of the acetabular cup inserter may be unobscured by the orientation sensor. The docking port 52 may be positioned to either the left or the right of the clamp 65.

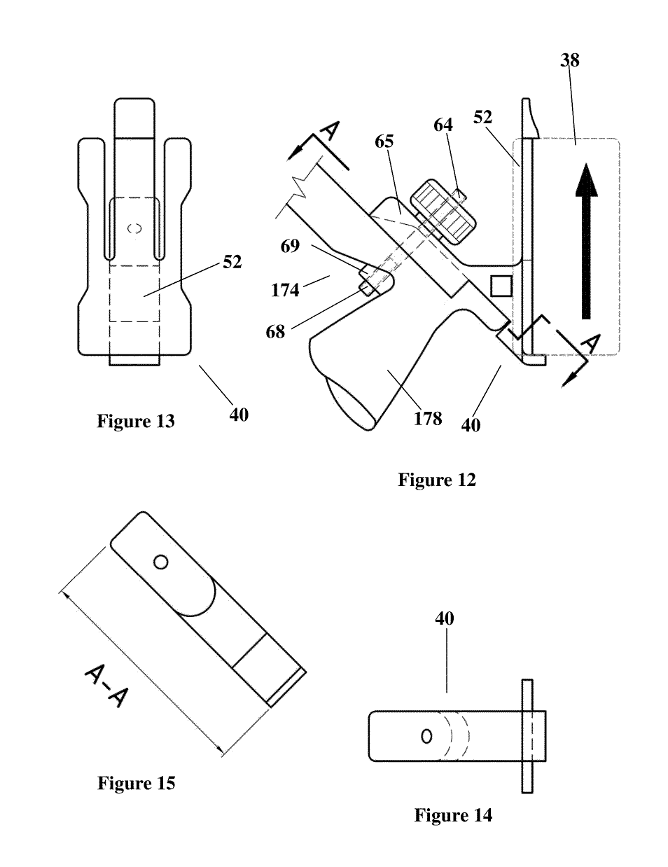

Another mounting 40 is illustrated in FIGS. 12-15, and in this case the mounting 40 is mounted to a surgical device, an acetabular reamer 170 (for example, as illustrated in FIG. 23). Turning to FIG. 23, the acetabular reamer 170 includes a cutting blade 172, an acetabular reamer driver 174, and a motor 176. The acetabular reamer 170 has a slot next to a handle 178 (the handle 178 forms part of the acetabular reamer driver 174). The mounting 40 (as illustrated in FIGS. 12-15 and 23) is intended to be mounted to an acetabular reamer driver 174 similar to the DePuy angled reamer driver catalogue number 920010031.

Returning to FIGS. 12-15, the acetabular reamer driver 174 has a slot next to a handle 178 through which a fastener may pass. The fastener may include a knurled nut 64, a bolt 68, and a nut 69. This mounting 40 includes a docking port 52 which is complementary in shape to sensing portion 38. Bending the tongue of the docking port 52 allows the sensing portion 38 to be uncoupled. The mounting 40 is arranged for quick acting positive position locking of the sensing portion 38.

This mounting may be made from a sterilisable material, and the body of the mounting 40 may be made of stainless steel. The mounting is especially made of a non-magnetisable material.

FIG. 23 illustrates an orientation sensor sensing portion 38 mounted to mounting 40, which is in turn mounted to acetabular reamer driver 174. The mounting 40 and sensing portion 38 illustrated in FIG. 23 is similar to that shown in FIGS. 12-15.

A further surgical device, an acetabular reamer 170, is illustrated in FIG. 25. In this acetabular reamer 170, the acetabular reamer driver 174 (as illustrated in FIG. 23) is replaced with mounting 40. The mounting 40 includes a sleeve 180, a handle 178, docking port 52 and bracket 168 for holding a monitor 10 and communicator 12. Sleeve 180, handle 178 and docking port 52 are integrally formed. In FIG. 25, the monitor 10 and communicator 12 are co-located in a tablet (or an iPad®) or a smartphone (such as an iPhone®) or the like. In use, the tablet or smartphone may be encased within a sterilisable housing which is held in, or forms part of the bracket 168. Furthermore, the sensing portion 38 is screwed into the docking port 52.

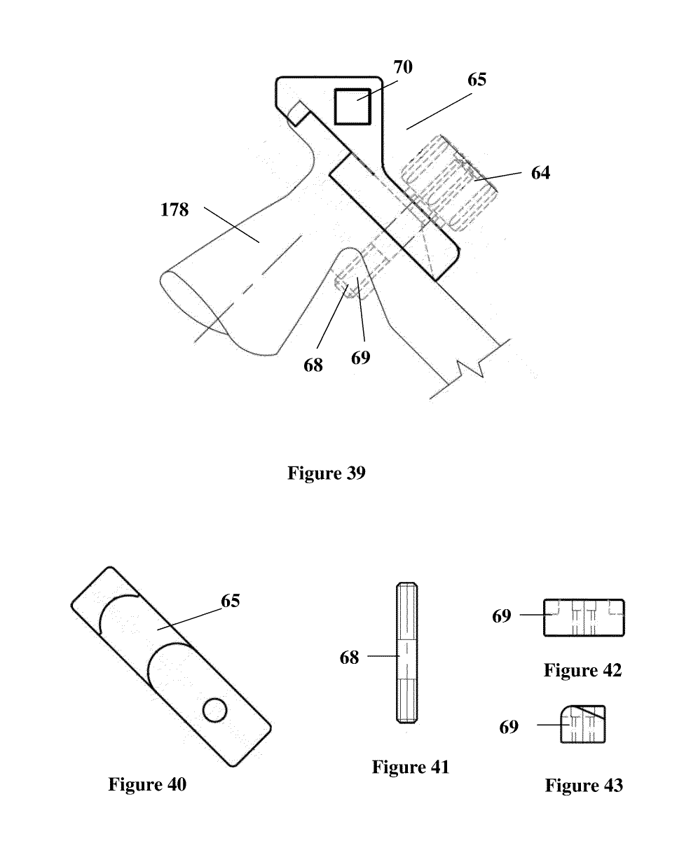

A further mounting 40 for an acetabular reamer 170 is illustrated in FIGS. 26-33 and 39-43. This mounting includes a clamp 65 (see FIG. 39) and a spacer attachment region 70. The clamp 65 includes a fastener which comprises a knurled nut 64 (see FIG. 39), a bolt 68 (see FIGS. 39 and 41), and a nut 69 (see FIGS. 39, 42 and 43). A first end 73 of spacer 72 (see FIGS. 28 and 29) may be inserted into spacer attachment region 70, and secured in place via clamp nut 74 (see FIGS. 26 and 27). A second end 75 of spacer 72 may be inserted into docking port attachment region 76 (see FIG. 31) and secured in place with clamp bolt 77 (see FIGS. 32 and 33). Orientation sensor sensing portion 38 (as shown in FIGS. 4 and 5) may be mounted to docking port 52 (see FIGS. 30 and 31). Advantageously, in this arrangement a surgeon's line of sight down the central axis of the acetabular cup inserter may be unobscured by the orientation sensor. The docking port 52 may be positioned to either the left or the right of the clamp 65.

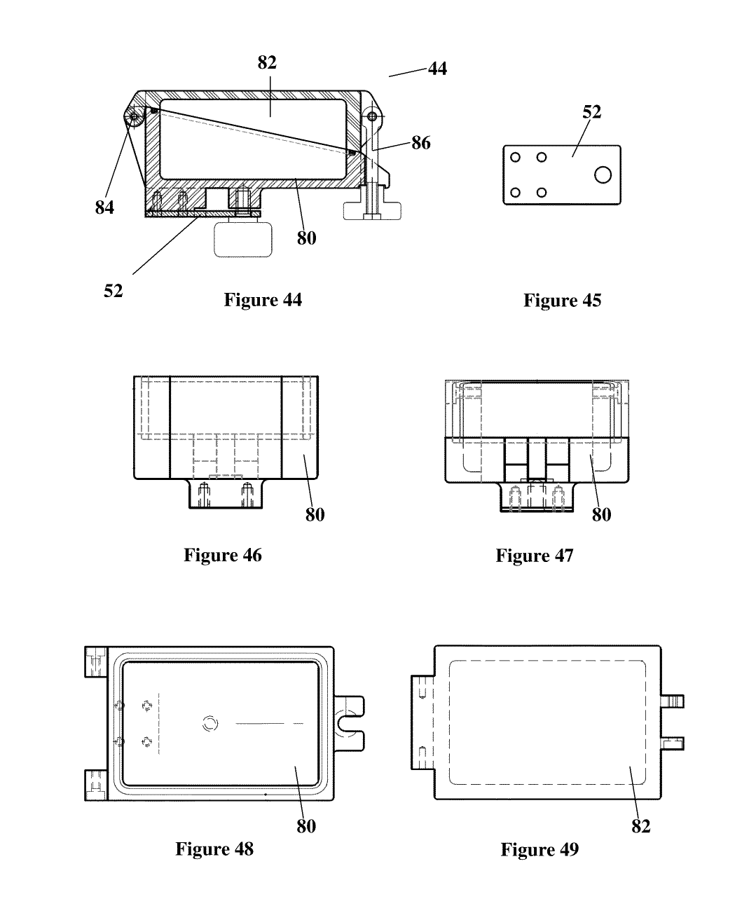

FIGS. 44 to 49 illustrate an orientation sensor sensing portion housing 44 according to a further example of the present invention. This housing 44 includes a body 80 and a lid 82, the lid being openable by rotation about hinge 84. The housing 44 also includes releasable fastener 86 (comprising a pivotable bolt, at the end of which is a nut which may be screwed onto the bolt to secure the lid shut). The housing 44 may be screwed onto docking port 52 (see FIGS. 44 and 45). Docking port 52 may be positioned, for example, within orientation sensor mounting 40 (instead of the slidable configuration illustrated in FIGS. 8-15, 30 and 31). This housing 44 may be sterilisable, and may be used to accommodate, for example, sensor 46 and power supply 42 (which may be unsterilised). This housing 44 may also be adapted to accommodate sensor 46, monitor 10 and communicator 12.

The housing 44 illustrated in FIGS. 44 to 49 may also be used as a patient sensor housing 34. In this case, the docking port 52 illustrated in FIGS. 44 and 45 may positioned on the patient sensor base 8 (instead of the slidable configuration illustrated in FIGS. 16-19 and 50-52). This housing 34 may be sterilisable and may be used to accommodate, for example, sensor 36 and power supply 32 (which may be unsterilised). This housing 34 may also be adapted to accommodate sensor 36, monitor 10 and communicator 12.

The surgical system 1 may further comprise a cradle for calibrating the sensors 36, 46. The cradle may accommodate sensors 36, 46 so that they are parallel to each other. In one embodiment, the cradle is configured to accommodate the sensing portions 6, 38 of the orientation sensor 4 and patient sensor 2, especially such that the sensing portions 6, 38 are parallel to each other.

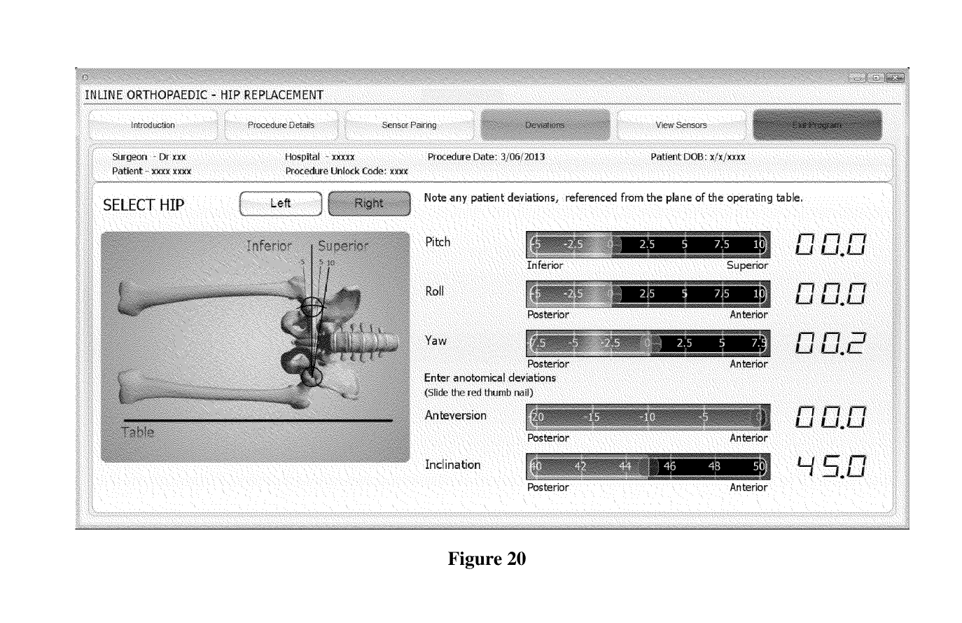

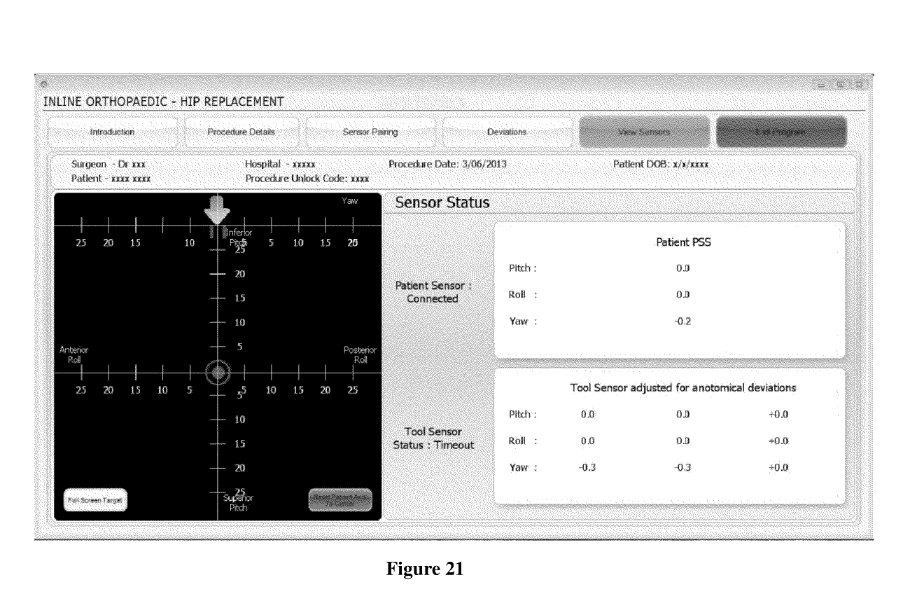

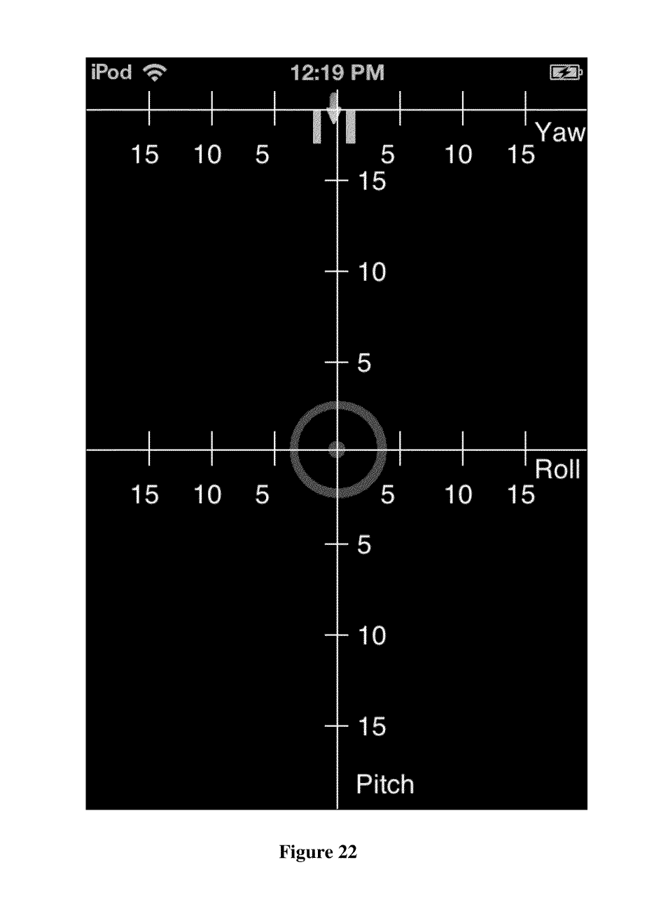

The surgical system illustrated in FIGS. 4-19 and 23-25 may be used according to the following steps:
1. Place the patient face down on the operating table, and draw posterior lines to mark the position for placing the base 8 of the patient sensor 2 (as illustrated in FIGS. 16-19);
2. Place and tape the base 8 to the patient (using surgical grade Elastoplast® tape, for example), preferably with the patient in the face down position;
3. Assemble and make ready an appropriate pelvic support brace and set the patient ready for covering;
4. Insert both sensors 36, 46 and batteries 32, 42 into their sterilised housings 34, 44 to form sensing portions 6, 38. Check to make sure that the full power light shows, if not replace the batteries. Place both sensing portions 6, 38 in the pairing cradle in the position marked and then place the loaded cradle anywhere on the operating table such that the indicating arrows (as illustrated in FIGS. 5 and 7) point to the head end of the table. The long axis of the cradle should be placed parallel to or as near to the longitudinal axis of the operating table as possible;
5. Start software using monitor 10 (computer (including a tablet or iPad® hard-drive)) and communicator 12 (screen);
6. Enter details of the procedure, including the surgeon's name, the patient's name, the hospital name, the date of birth of the patient and the date of the procedure;
7. Pair (calibrate) the sensors, such that the pitch, roll and yaw for both sensors are substantially zero. The calibration step may be performed in a matter of seconds;
8. Mount the patient sensor sensing portion 6 to the base 8, and mount the orientation sensor sensing portion 38 to or relative to the surgical device (for example, mount the sensing portion 38 to mounting 40, which may be mounted to a surgical device 16/170 or a placement device 48 as illustrated in FIGS. 8-15 and 23-25);
9. Adjust the optimal orientation of the device, as recorded on the computer, to account for any differences in pitch, roll or yaw due to the position of the patient on the operating table. Also enter any anatomical deviations specific to the patient, such as the patient's pelvic tilt angle (as measured by anteversion and inclination) (see FIG. 20);
10. View the orientation of the surgical device relative to the patient's anatomy, as illustrated in FIGS. 21 and 22;
11. Perform the acetabular reaming or acetabular cup placement operation as described above; and
12. A record of the output of the surgical system during the operation may be stored on a memory card for future reference.

### Example

A confidential trial ably illustrated that the system provides a simple means of enabling a surgeon to ream and place the acetabular cup in the appropriate position with respect to the predetermined angles of inversion and inclination and relative to the patient as a consequence of the ongoing automatic compensation for patient movement. In this trial, the patient sensor, orientation sensor, surgical devices, monitors and communicators were those discussed above in relation to FIGS. 4-22.

During the trial, the orientation sensor was subjected to the normal amount of shock forces generated by hammering the cup into position. This had zero impact on the functioning of the orientation sensor.

The sensors were built using high quality electronic components. Under hospital theatre conditions, testing have proven the roll and pitch is within plus or minus 0.5 degree for a range of plus or minus 20 degrees and the yaw is within plus or minus 1.0 degree for a range of plus or minus 10 degrees.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

### ADVANTAGES OF THE INVENTION

The present invention, in various embodiments, provides an accurate, easy to use alignment device in various surgical operations such as total hip replacement surgery. The device may be adaptable to most prostheses.

The present invention, in various embodiments, may provide the following advantages:

For surgeons: allows for improved accuracy with patient benefits, enables greater number of minimally invasive surgical operations and less need for revision surgery, allows for improved surgical outcomes, easier operation for surgeons who perform relatively few total hip replacement operations, and a standardisation of the total hip replacement procedure;

For patients: reduces trauma, allows for faster recovery and improved surgical outcomes, and reduces the likelihood of revision surgery; and

For healthcare funders: expected to reduce the total cost of the total hip replacement procedure, reduce the likelihood of revision surgery, and increase the operating life of the prosthesis.

### CITATION LIST

International Publication No. WO2010/031111
International Publication No. WO2004/112610
Langton, D. J. et al. (2011), J Bone Joint Surg[Br] 93-B:164-71.
Katz, J. N. (2007) The Orthopaedic Journal at Harvard Medical School 9:101-106.
Kurtz, S. M. (2010) Paper #365. Presented at the 56th Annual Meeting of the Orthopaedic Research Society. Mar. 6-9, 2010. New Orleans.

1-88. (canceled)

89. A surgical system for orienting a surgical device relative to a patient's anatomy, the system comprising:
a. a patient sensor for sensing the orientation of the patient's anatomy, wherein the patient sensor is mountable on the patient's anatomy, and wherein when the patient's anatomy is the pelvis the patient sensor does not include a pelvic brace for securing the patient to an operating table;
b. an orientation sensor for sensing the orientation of a surgical device;
c. a monitor for monitoring the orientation of the surgical device relative to the sensed patient's anatomy; and
d. a communicator for guiding the monitored surgical device to an optimal surgical device orientation relative to the sensed patient's anatomy;
wherein the patient's anatomy is the pelvis or the scapula.

90. The system of claim 89, wherein the monitor determines the optimal surgical device orientation relative to the sensed patient's anatomy by considering natural variations in the patient's anatomy and the orientation of the patient's anatomy sensed by the patient sensor.

91. The system of claim 89, wherein the monitor determines the difference between the optimal surgical device orientation relative to the sensed patient's anatomy and the orientation of the surgical device relative to the sensed patient's anatomy, and wherein the communicator communicates the difference between the optimal surgical device orientation relative to the sensed patient's anatomy and the orientation of the surgical device relative to the sensed patient's anatomy.

92. The system of claim 89, wherein the system is for surgery on the hip or shoulder joint of a patient.

93. The system of claim 89, wherein the system is for use in total hip replacement surgery.

94. The system of claim 89, wherein the patient sensor comprises a base and a sensing portion mountable to the base, wherein the base comprises a fastener for mounting the base on the patient's anatomy.

95. The system of claim 89, wherein the patient's anatomy is the pelvis.

96. The system of claim 95, wherein the monitor determines the optimal surgical device orientation by considering the patient's measured pelvic tilt angle.

97. The system of claim 95, wherein the patient sensor is configured to be aligned with at least one of the patient's iliac crest, sacrum and pubic symphysis.

98. The system of claim, wherein the patient sensor is configured to be mounted on at least one of the patient's iliac crest, sacrum and pubic symphysis.

99. The system of claim 89, wherein the orientation sensed by the patient sensor and the orientation sensor is pitch, roll and yaw.

100. The system of claim 89, wherein:
the patient sensor comprises at least one sensor for sensing the orientation of the patient's anatomy, wherein the at least one sensor is selected from the group consisting of a gyroscope, a magnetometer, an accelerometer, an inclinometer and an inertial sensor; and
the orientation sensor comprises at least one sensor for sensing the orientation of the surgical device, wherein the at least one sensor is selected from the group consisting of a gyroscope, a magnetometer, an accelerometer, an inclinometer and an inertial sensor.

101. The system of claim 89, wherein the system comprises two patient sensors.

102. The system of claim 89, further comprising a surgical device for surgical use on the patient's anatomy.

103. The system of claim 89, wherein the surgical device is a reamer or a prosthetic component, and wherein the orientation sensor is mountable to the reamer or mountable to a placement device for placing the prosthetic component.

104. The system of claim 103, wherein the orientation sensor comprises a sensing portion and a mounting for mounting the orientation sensor to the reamer or to the placement device, wherein the sensing portion is mountable to the mounting, and wherein the mounting comprises one or more of a docking port for mounting the sensing portion, a shock absorber, and a fastener for fastening the orientation sensor to a reamer or placement device.

105. The system of claim 102, wherein the surgical device is an acetabular reamer and the patient's anatomy is the pelvis.

106. The surgical system of claim 105, wherein the system is configured to electronically ameliorate the effect of the acceleration of the reamer on the orientation sensor.

107. A surgical system for monitoring the orientation of a surgical device relative to a patient's anatomy, the system comprising:
a. a patient sensor for sensing the orientation of the patient's anatomy, wherein the patient sensor is mountable on the patient's anatomy, and wherein when the patient's anatomy is the pelvis the patient sensor does not include a pelvic brace for securing the patient to an operating table;
b. an orientation sensor for sensing the orientation of a surgical device; and
c. a monitor for monitoring the orientation of the surgical device relative to the sensed patient's anatomy;
wherein the patient's anatomy is the pelvis or the scapula.
